(19) United States
(12) Patent Application Publication
(10) Pub. No.: US 2026/0346173 A1
(43) Pub. Date: Nov. 5, 2026

(54) CELLULOSE NANOFIBER-BASED COMPOSITIONS FOR EHNANCED FILTRATION EFFICIENCY AND METHODS OF USE THEREOF

(71) Applicant: Board of Regents, The University of Texas System, Austin, TX (US)

(72) Inventors: Jonathan Chen, Austin, TX (US); Sophia Jackson, Austin, TX (US)

(21) Appl. No.: 19/118,203

(22) PCT Filed: Oct. 3, 2023
(86) PCT No.: PCT/US2023/075819

§ 371 Date: Apr. 3, 2025

Related U.S. Application Data
(60) Provisional application No. 63/378,138, filed on Oct. 3, 2022.
(60) Provisional application No. 63/513,431, filed on Jul. 13, 2023.

(51) Int. Cl. B01D 39/18; A41D 13/11; A61K 31/722; A62B 23/02; B01D 46/00; B01D 46/54

(52) U.S. Cl. CPC B01D 39/18; A41D 13/1192; A61K 31/722; B01D 46/0028; B01D 46/546; A62B 23/025; B01D 2239/025; B01D 2239/0266; B01D 2239/0442; B01D 2239/0478; B01D 2239/08; B01D 2239/10; B01D 2279/65

(57) ABSTRACT

The present invention provides a composition for increasing filtration efficiency comprising cellulose nanofiber (CNF) or a derivative thereof, an adhering agent (e.g., a polyhydroxy polymer, such as polyvinyl alcohol (PVA)), and a carrier (e.g., aqueous solvent). In various embodiments, the composition increases barrier properties against particles (e.g., aerosol particles). In some embodiments, the composition breaks a membrane of at least one cell of interest (e.g., pathogenic cell). In one aspect, the present invention provides a device comprising said composition and a material of interest (e.g., textile material). In some embodiments, the textile material is a mask, clothing, or filter. In one aspect, the present invention provides a method of increasing barrier properties of a material of interest against at least one particle using said composition.

### CROSS-REFERENCE TO RELATED APPLICATIONS

This application claims priority to U.S. Provisional Application Ser. No. 63/513,431, filed Jul. 13, 2023, and U.S. Provisional Application Ser. No. 63/378,138, filed Oct. 3, 2022, the disclosures of which are hereby incorporated by reference herein in their entireties.

### BACKGROUND OF THE INVENTION

The onset of the Covid-19 pandemic in December 2019 had a profound effect on individuals around the world. It has disrupted normal routines and led to unprecedented measures, such as lockdowns and mask mandates. One of the biggest factors in the pandemic is the usage of face-covering masks as an extended form of Personal Protective Equipment (PPE) (Tessarolo et al., 2021, International journal of Environmental Research and Public Health, 18:1462). This is to protect the wearer and surrounding others because SARS-CoV-2 is primarily transmitted between people through respiratory droplets and contact routes (Chaussade et al., 2021, Endoscopy International Open, 9:E482). Facial masks reign as one of the first lines of defense (as well as vaccination) against easily transmittable Covid-19. Luckily through recent years, various scientific endeavors in the textile industry have enriched textiles with properties (like improved filtration, antibacterial and antiviral activity, breathability, etc.) crucial for the successful prevention of the spread of infectious diseases (Ivanoska-Dacikj et al., 2020, Reviews on Advanced Materials Science, 59:487-505). Masks have become the new normal, worn in private places, public places, and other heavily populated areas. One of the most easily attainable types of masks for the common public are standard surgical masks. These masks are constructed with layered melt-blown nonwovens made of thermoplastic polypropylene, which is not biodegradable.

Even though masks are efficient, they are made for limited usage, making them nearly non-reusable, causing an exponential amount of disposal waste. New research found that across eleven countries studied, the number of masks that ended up as litter/waste increased 84-fold from pre-pandemic levels (Norris et al., 2021, Medical News Today, COVID-19: Research unmasks the environmental impact of PPE). This increase in mask production has led to exponential waste levels, such as up to 7,200 tons of medical-type waste daily during the Covid-19 pandemic, and a primary cause of this waste is disposable masks (Trafton et al., 2021, MIT News on Campus and Around the World, The Environmental Toll of Disposable Masks: A new study calculates the waste generated by N95 usage and suggests possible ways to reduce it).

To promote availability and sustainability during the pandemic, many companies have taken stride in constructing facial masks out of various textiles, inspiring citizens at home to create their own Do-It-Yourself (DIY) masks. These masks are constructed of natural and synthetic textiles and allow for multi-use wearing through at-home laundering. With the high demand for facial protection in the present day, DIY masks are the most sustainable application option. One detriment of selecting at-home textiles relates to their porous sizes being too large to withhold the entirety of aerosol particles. Previous studies have also reported that the virus can survive on cloth or standard textiles for several days (Ivanoska-Dacikj et al., 2020, Reviews on Advanced Materials Science, 59:487-505).

Thus, there is a need in the art for compositions that can enhance filtration efficiencies of various materials (e.g., textiles). The present invention addresses this need.

### BRIEF SUMMARY OF THE INVENTION

In one aspect, the present invention provides a composition for increasing filtration efficiency, wherein the composition comprises cellulose nanofiber (CNF) or a derivative thereof, an adhering agent, and a carrier.

In one embodiment, the composition comprises between about 0.01 wt % to about 20 wt % of CNF. In one embodiment, the composition comprises between about 0.06 wt % to about 3 wt % of CNF. In one embodiment, the composition comprises between about 0.01 wt % to about 20 wt % of the adhering agent. In one embodiment, the composition comprises between about 0.7 wt % to about 9 wt % of the adhering agent.

In one embodiment, the adhering agent comprises at least one polymer. In one embodiment, the polymer has a size of between about 10 kDa to about 100,000 kDa. In one embodiment, the polymer has a size of between about 80 kDa to about 130 kDa.

In one embodiment, the polymer is selected from the group consisting of a polyhydroxy polymer, water-soluble polymer, biodegradable polymer, biocompatible polymer, and any combination thereof. In one embodiment, the polymer is selected from the group consisting of a polyvinyl alcohol (PVA), polyethylene glycol (PEG), 1,2-distearoyl-sn-glycero-3-phosphoethanolamine-polyethylene glycol (DSPE-PEG), 1,2-distearoyl-sn-glycero-3-phosphoethanolamine-N-[amino (polyethylene glycol)-2000](DSPE-PEG (2000)-amine), and any combination thereof. In one embodiment, the polymer is PVA.

In one embodiment, the adhering agent specifically binds CNF to a surface of a material of interest. In one embodiment, the material of interest is selected from the group consisting of a textile material, paper, glass, metal, wood, plastic, or any combination thereof. In one embodiment, the textile material comprises polyester, polypropylene, polyethylene, polyurethane, polyacrylonitrile, cotton, linen, nylon, silk, rayon, modal, cellulose, wool, chenille, leather, spandex, or any combination thereof.

In one embodiment, the carrier comprises an aqueous solution. In one embodiment, the aqueous solution is an acidic solution, basic solution, or neutral solution. In one embodiment, the aqueous solution comprises water, methanol, ethanol, propanol, isopropyl alcohol, butyl alcohol, diethyl ether, acetone, methyl acetate, ethyl acetate, acetic acid, or any combination thereof.

In one embodiment, the composition is a water-soluble composition, biodegradable composition, biocompatible composition, and any combination thereof.

In one embodiment, the composition increases barrier properties against particles. In one embodiment, the particles have an average hydrodynamic diameter of about 0.1 nm to about 100,000 nm. In one embodiment, the particles are aerosol particles. In one embodiment, the aerosol particles comprise at least one selected from the group consisting of an aerosol bacterium, aerosol virus, aerosol pathogen, aerosol fungus, aerosol pollutant, aerosol toxin, aerosol allergen, aerosol particle associated with a disease or disorder, and any combination thereof.

In one embodiment, the composition breaks a membrane of at least one cell of interest. In one embodiment, the cell of interest is a pathogen cell.

In some embodiments, the composition is an antimicrobial composition, antibacterial composition, antiviral composition, antifungal composition, or any combination thereof.

In one embodiment, the composition further comprises at least one therapeutic agent. In one embodiment, the composition comprises between about 0.01 wt % to about 20 wt % of the at least one therapeutic agent.

In one embodiment, the composition comprises between about 0.1 wt % to about 2 wt % of the at least one therapeutic agent. In one embodiment, the at least one therapeutic agent is selected from the group consisting of antimicrobial agent, antibacterial agent, antiviral agent, antifungal agent, or any combination thereof.

In one embodiment, the at least one therapeutic agent is chitosan (CS) or a derivative thereof. In one embodiment, the composition comprises between about 0.01 wt % to about 20 wt % of CS or a derivative thereof. In one embodiment, the composition comprises between about 0.1 wt % to about 2 wt % of CS or a derivative thereof.

In one aspect, the present invention provides a method of increasing barrier properties of a material of interest against at least one particle, wherein the method comprises applying at least one layer of a composition for increasing filtration efficiency, wherein the composition comprises cellulose nanofiber (CNF) or a derivative thereof, an adhering agent, and a carrier, to the surface of the material of interest.

In one embodiment, the composition a) decreases a contact angle between the at least one particle and the material of interest; b) decreases a size of at least one pore of the material of interest; or c) a combination thereof.

In one embodiment, the particles have an average hydrodynamic diameter of about 0.1 nm to about 100,000 nm. In one embodiment, the particles are aerosol particles. In one embodiment, the aerosol particles comprise at least one selected from the group consisting of an aerosol bacterium, aerosol virus, aerosol pathogen, aerosol fungus, aerosol pollutant, aerosol toxin, aerosol allergen, aerosol particle associated with a disease or disorder, and any combination thereof.

In one embodiment, the material of interest is selected from the group consisting of a textile material, paper, glass, metal, wood, plastic, or any combination thereof. In one embodiment, the textile material comprises polyester, polypropylene, polyethylene, polyurethane, polyacrylonitrile, cotton, linen, nylon, silk, rayon, modal, cellulose, denim, wool, chenille, leather, spandex, or any combination thereof.

In one aspect, the present invention provides a method of applying at least one composition comprises cellulose nanofiber (CNF) or a derivative thereof, an adhering agent, and a carrier to a material of interest, wherein the method comprises applying at least one layer of the composition to the surface of the material of interest.

In one embodiment, the material of interest is selected from the group consisting of a textile material, paper, glass, metal, wood, plastic, or any combination thereof. In one embodiment, the textile material comprises polyester, polypropylene, polyethylene, polyurethane, polyacrylonitrile, cotton, linen, nylon, silk, rayon, modal, cellulose, denim, wool, chenille, leather, spandex, or any combination thereof.

In one embodiment, at least one layer of the composition is applied to the surface of the material of interest by spraying. In one embodiment, the composition is sprayed to the surface of the material of interest at least 5 times.

In one aspect, the present invention provides a device comprising a material of interest and a composition comprises cellulose nanofiber (CNF) or a derivative thereof, an adhering agent, and a carrier, wherein the device comprises at least one layer of the composition coated on the surface of the material of interest.

In one embodiment, the material of interest is selected from the group consisting of a textile material, paper, glass, metal, wood, plastic, or any combination thereof. In one embodiment, the textile material comprises polyester, polypropylene, polyethylene, polyurethane, polyacrylonitrile, cotton, linen, nylon, silk, rayon, modal, cellulose, denim, wool, chenille, leather, spandex, or any combination thereof.

In one embodiment, the device increases barrier properties against particles.

In one embodiment, the device is a mask, clothing, fabric, textile, furniture, carpet, curtain, upholstery, filter, or any combination thereof.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a schematic representation of a DIY-based fabric mask solution.

FIG. 2 depicts a schematic representation of the biomimic concept of nanoscale pillars rupturing bacterial cells.

FIG. 3, comprising FIG. 3A and FIG. 3B, depicts representative video contact angle (VCA) Optima XE photos of cellulose nanofiber/polyvinyl alcohol (CNF/PVA) water-based dispersion on nylon fabric vs control. FIG. 3A depicts a representative VCA Optima XE photo of a control nylon fabric. FIG. 3B depicts a representative VCA Optima XE photo of CNF/PVA water-based dispersion on nylon fabric.

FIG. 4, comprising FIG. 4A and FIG. 4B, depicts representative Launder-O-Meter tested samples of cotton #1, silk, polyester, nylon, and polypropylene fabrics captured pre-laundering. FIG. 4A depicts representative Launder-O-Meter tested samples of cotton #1, silk, and polyester fabrics captured pre-laundering. FIG. 4B depicts representative Launder-O-Meter tested samples of nylon and polypropylene fabrics captured pre-laundering.

FIG. 5, comprising FIG. 5A and FIG. 5B, depicts representative Launder-O-Meter tested samples of cotton #1, silk, polyester, nylon, and polypropylene fabrics captured post-laundering. FIG. 5A depicts representative Launder-O-Meter tested samples of nylon and polypropylene fabrics captured post-laundering. FIG. 5B depicts representative Launder-O-Meter tested samples of cotton #1, silk, and polyester fabrics captured post-laundering.

FIG. 6 depicts representative results of two-way ANOVA analysis of VCA contact angle measurements.

FIG. 7, comprising FIG. 7A through FIG. 7J, depicts representative control and experimental photos of CNF/PVA water-based dispersion on various textiles. FIG. 7A depicts a representative photo of a cotton #1 control at 10×. FIG. 7B depicts a representative photo of CNF/PVA water-based dispersion on cotton #1 at 10×. FIG. 7C depicts a representative photo of a polyester control at 10×. FIG. 7D depicts a representative photo of CNF/PVA water-based dispersion on polyester at 10×. FIG. 7E depicts a representative photo of a nylon control at 40×. FIG. 7F depicts a representative photo of CNF/PVA water-based dispersion on nylon at 40×. FIG. 7G depicts a representative photo of a polypropylene control at 10×. FIG. 7H depicts a representative photo of CNF/PVA water-based dispersion on polypropylene at 10×. FIG. 7I depicts a representative photo of a silk control at 20×. FIG. 7J depicts a representative photo of CNF/PVA water-based dispersion on silk at 20×.

FIG. 8, comprising FIG. 8A through FIG. 8J, depicts representative FEI Quanta 650 scanning electron microscope (SEM) images of various textile swatches layered with CNF/PVA water-based dispersion spray. FIG. 8A depicts a representative SEM image of a cotton #1 layered with CNF/PVA water-based dispersion spray at 500×. FIG. 8B depicts a representative SEM image of a cotton #1 layered with CNF/PVA water-based dispersion spray at 3000×. FIG. 8C depicts a representative SEM image of a polyester layered with CNF/PVA water-based dispersion spray at 500×. FIG. 8D depicts a representative SEM image of a polyester layered with CNF/PVA water-based dispersion spray at 3000×. FIG. 8E depicts a representative SEM image of a nylon layered with CNF/PVA water-based dispersion spray at 500×. FIG. 8F depicts a representative SEM image of a nylon layered with CNF/PVA water-based dispersion spray at 3000×. FIG. 8G depicts a representative SEM image of a polypropylene layered with CNF/PVA water-based dispersion spray at 500×. FIG. 8H depicts a representative SEM image of a polypropylene layered with CNF/PVA water-based dispersion spray at 3000×. FIG. 8I depicts a representative SEM image of a silk layered with CNF/PVA water-based dispersion spray at 3000×. FIG. 8J depicts a representative SEM image of a silk layered with CNF/PVA water-based dispersion spray at 3000×.

FIG. 9, comprising FIG. 9A and FIG. 9B, depicts representative results demonstrating Dry-Up/Wet-Up curves on cotton 2 fabric between control and CNF/PVA spray. FIG. 9A depicts representative results demonstrating Dry-Up curves on cotton 2 fabric between control and CNF/PVA spray. FIG. 9B depicts representative results demonstrating Wet-Up curves on cotton 2 fabric between control and CNF/PVA spray.

FIG. 10 depicts representative results demonstrating bacterial filtration efficiency regarding ASTM F2100-19 specification requirements.

FIG. 11 depicts representative SEM microscope photos of a control nylon fabric, nylon fabric sprayed with CNF, and nylon fabric sprayed with CNF/PVA after 40× magnification, HDR 3-layered, and differential image contract.

FIG. 12 depicts representative growth rate, from backscatter data, of E. Coli under control, CNF, CNF and CS, and silver nitrate conditions.

FIG. 13 depicts representative growth rate, from OD600 measurements, of E. Coli under control, CNF, CNF and CS, and silver nitrate conditions.

FIG. 14 depicts representative growth rate, from backscatter data, of E. Coli under control and CNF conditions.

FIG. 15 depicts representative growth rate, from OD600 measurements, of E. Coli under control and CNF conditions.

FIG. 16 depicts representative solutions comprising E. Coli under control, CNF, CNF and CS, and silver nitrate conditions depicted in FIG. 12 and FIG. 13 (left) and E. Coli under control, CNF, and CNF conditions depicted in FIG. 14 and FIG. 15 (right).

FIG. 17 depicts representative growth rate, from backscatter data, of E. Coli under control and CNF+CS conditions.

FIG. 18 depicts representative growth rate, from OD600 measurements, of E. Coli under control and CNF+CS conditions.

FIG. 19 depicts representative agar plates of E. coli before (top) and after (center and bottom) incubation. Left column=control, middle column=CNF, and right column=CNF and CS.

### DETAILED DESCRIPTION

The present invention is based, in part, on the unexpected discovery that applying a composition comprising cellulose nanofiber (CNF) and polyvinyl alcohol (PVA) to the surface of a textile material increased the filtration efficiency of the textile material. Thus, in one aspect, the present invention provides a composition for increasing filtration efficiency comprising CNF or a derivative thereof, an adhering agent (e.g., a polyhydroxy polymer, such as PVA), and a carrier (e.g., aqueous solvent).

In various embodiments, the composition increases barrier properties against particles (e.g., aerosol particles, such as an aerosol bacterium, aerosol virus, aerosol pathogen, aerosol fungus, aerosol pollutant, aerosol toxin, and/or aerosol particle associated with a disease or disorder).

In some embodiments, the composition breaks a membrane of at least one cell of interest (e.g., pathogenic cell, such as a bacterium cell, viral cell, fungal cell, etc.).

In one aspect, the present invention provides a device comprising said composition and a material of interest (e.g., textile material, such as polyester, polypropylene, polyethylene, polyurethane, polyacrylonitrile, cotton, linen, nylon, silk, rayon, modal, cellulose, denim, wool, chenille, leather, and/or spandex). In some embodiments, the textile material is a mask, clothing, or filter.

In one aspect, the present invention provides a method of increasing barrier properties of a material of interest against at least one particle using the composition of the present invention.

In another aspect, the present invention provides a method of applying at least one composition of the present invention to a material of interest.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, +5%, +1%, or ±0.1% from the specified value, as such variations are appropriate to perform the disclosed methods. For example, “about 40 [units]” may mean within ±25% of 40 (e.g., from 30 to 50), within +20%, +15%, +10%, 9%, 8%, ±7%, +6%, +5%, +4%, +3%, +2%, +1%, less than +1%, or any other value or range of values therein or therebelow. Furthermore, the phrases “less than about [a value]” or “greater than about [a value]” should be understood in view of the definition of the term “about” provided herein.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient (s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, corn starch, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly (vinylchloride), micro-porous polyamides, microporous modacrylic copolymers, microporous styrene-acrylic and its copolymers); porous polysulfones, halogenated poly (vinylidene), polychloroethers, acetal polymers, polyesters prepared by esterification of a dicarboxylic acid or anhydride with an alkylene polyol, poly (alkylenesulfides), phenolics, polyesters, asymmetric porous polymers, cross-linked olefin polymers, hydrophilic microporous homopolymers, copolymers or interpolymers having a reduced bulk density, and other similar materials, poly (urethane), cross-linked chain-extended poly (urethane), poly (mides), poly (benzimidazoles), collodion, regenerated proteins, semi-solid cross-linked poly (vinylpyrrolidone).

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

The terms “coat,” “coated,” or “coating,” as used herein, refer to at least a partial coating of a material of interest (e.g., a textile material). One hundred percent coverage is not necessarily implied by these terms.

The term “binding” refers to a direct association between at least two molecules or at least two components, due to, for example, covalent, electrostatic, hydrophobic, ionic and/or hydrogen-bond interactions.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components. Alternatively, molecules or components thereof may be contacted combining a fluid component with molecules immobilized on or in a cell or on or in a substrate, such as a polymer bead, a membrane, a polymeric glass substrate or substrate surface derivatized to provide immobilization of target molecules, candidate molecules, competitive binding reference molecules or any combination of these. Molecules or components thereof may be contacted by selectively adjusting solution conditions such as, the composition of the solution, ion strength, pH or temperature. Molecules or components thereof may be contacted in a static vessel, such as a microwell of a microarray system, or a flow-through system, such as a microfluidic or nanofluidic system. Molecules or components thereof may be contacted in or on a variety of cells, media, liquids, solutions, colloids, suspensions, emulsions, gels, solids, membrane surfaces, glass surfaces, polymer surfaces, vesicle samples, bilayer samples, micelle samples and other types of cellular models or any combination of these. As used herein, the term “contacting” includes, but is not limited to, impregnating, compounding, mixing, integrating, coating, rubbing, painting, spraying, immersing, rolling, smearing and dipping.

As used herein, the term “antimicrobial” refers to an ability to kill or inhibit the growth of microorganisms, including but not limited to bacteria, viruses, yeast, fungi, and protozoa, or to attenuate the severity of a microbial infection. The antimicrobial compounds or compositions of the present disclosure are compounds or compositions that may be used for cleaning or sterilization, or may be used in the treatment of disease and infection. The applications may include both in vitro and in vivo antimicrobial uses. “Applying” an antimicrobial composition may include administrating a composition into a human or animal subject.

The term “bacterium”, as used herein, refers to “pathogenic bacterium” and/or “non-pathogenic bacterium”. For example, the term “non-pathogenic bacterium” refers to bacterium that is not capable of causing disease or harmful responses in a host. In some embodiments, bacteria are commensal bacteria. Examples of bacteria include, but are not limited to Escherichia coli LF82, Enterococcus faecalis, Lactobacillis plantarum, Faecalibacterium prauznitzii, Bifidobacterium longum, Bacteroides vulgatus, Ruminococcus gnavus, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium in antis, Bifidobacterium lactis, Bifidobacterium longum, Clostridium butyricum, Enterococcus aecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Saccharomyces boulardii, and Brevundimonas diminuta (Sonnenborn et al., 2009; Dinleyici et al., 2014; U.S. Pat. Nos. 6,835,376; 6,203,797; 5,589,168; 7,731,976). In some embodiments, naturally pathogenic bacteria may be genetically engineered to provide reduce or eliminate pathogenicity.

The term “virus” as used herein is defined as a particle consisting of nucleic acid (RNA or DNA) enclosed in a protein coat, with or without an outer lipid envelope, which is capable of replicating within a whole cell. Examples of virus include, but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picomaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

The term “allergen” as used herein refers to any molecule capable of inducing allergy, i.e. IgE mediated reactions upon repeated exposure to an allergen, including seasonal allergy. Examples of allergens include, but are not limited to, pollen allergens (tree, weed, herb and grass pollen), mite allergens (from e.g. house dust mites and storage mites), insect allergens (e.g. inhalant origin allergens), animal allergens (from e.g. hair, dander feathers from e.g. dog, cat, horse, rat, mouse, guinea pig, rabbit, bird), fungal or mold allergens. Examples of pollen allergens include, but are not limited to, tree pollen, including pollen from trees belonging to the orders Fagales, Lamiales, Proteales, Pinales, Fabales, Malpighiales, Sapindales, Myrtales, Rosales and Arecales; weed pollen, including pollen from weeds belonging to the families Asteraceae, Amaranthaceae, Plantaginaceae, Uritaceae and Euphorbiaceae; grass pollen, including pollen from grass belonging to the genera Oryza, Phragmites, Cynodon, Paspalum, Sorghum, Zea, Dactylis, Festuca, Lolium, Poa, Anthoxanthym, Avena, Holcus, Phalaris, Agrostis, Alopecurus, Phleum, Bromus, Hordeum, Secale, and Triticum; microbial species, e.g. bacteria and fungi, that colonize pollen; and pollen-derived submicronic and paucimicronic particles.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery that applying a composition comprising CNF and PVA to the surface of a textile material increased the filtration efficiency of the textile material. Thus, in one aspect, the present invention provides a composition for increasing filtration efficiency comprising CNF or a derivative thereof, an adhering agent (e.g., a polyhydroxy polymer, such as PVA), and a carrier (e.g., aqueous solvent).

In various embodiments, the composition increases barrier properties against particles (e.g., aerosol particles, such as an aerosol bacterium, aerosol virus, aerosol pathogen, aerosol fungus, aerosol pollutant, aerosol toxin, and/or aerosol particle associated with a disease or disorder).

In some embodiments, the composition breaks a membrane of at least one cell of interest (e.g., pathogenic cell, such as a bacterium cell, viral cell, fungal cell, etc.).

In one aspect, the present invention provides a device comprising said composition and a material of interest (e.g., textile material, such as polyester, polypropylene, polyethylene, polyurethane, polyacrylonitrile, cotton, linen, nylon, silk, rayon, modal, cellulose, denim, wool, chenille, leather, and/or spandex). In some embodiments, the textile material is a mask, clothing, or filter.

In one aspect, the present invention provides a method of increasing barrier properties of a material of interest against at least one particle using the composition of the present invention.

In another aspect, the present invention provides a method of applying at least one composition of the present invention to a material of interest.

### Compositions

In one aspect, the present invention provides a composition for increasing filtration efficiency. In another aspect, the present invention provides a composition having antimicrobial, antibacterial, antiviral, and/or antifungal properties. Thus, in various embodiments, the composition is an antimicrobial composition, antibacterial composition, antiviral composition, antifungal composition, or any combination thereof.

In various embodiments, the composition comprises CNF or a derivative thereof. In some embodiments, the composition comprises CNF or a derivative thereof and an adhering agent. In some embodiments, the composition comprises CNF or a derivative thereof, an adhering agent, and a carrier.

In one embodiment, the composition is a homogenous composition. In one embodiment, the composition is a heterogeneous composition.

In one embodiment, the composition is an emulsion. In one embodiment, the composition is a dispersion.

In some embodiments, the composition comprises between about 0.01 wt % to about 50 wt % of CNF or the derivative thereof. In some embodiments, the composition comprises between about 0.01 wt % to about 40 wt % of CNF or the derivative thereof. In some embodiments, the composition comprises between about 0.01 wt % to about 30 wt % of CNF or the derivative thereof. In some embodiments, the composition comprises between about 0.01 wt % to about 20 wt % of CNF or the derivative thereof. In some embodiments, the composition comprises between about 0.01 wt % to about 10 wt % of CNF or the derivative thereof. In some embodiments, the composition comprises between about 0.01 wt % to about 9 wt % of CNF or the derivative thereof. In some embodiments, the composition comprises between about 0.01 wt % to about 8 wt % of CNF or the derivative thereof. In some embodiments, the composition comprises between about 0.01 wt % to about 7 wt % of CNF or the derivative thereof. In some embodiments, the composition comprises between about 0.01 wt % to about 6 wt % of CNF or the derivative thereof. In some embodiments, the composition comprises between about 0.01 wt % to about 5 wt % of CNF or the derivative thereof. In some embodiments, the composition comprises between about 0.01 wt % to about 4 wt % of CNF or the derivative thereof. In some embodiments, the composition comprises between about 0.01 wt % to about 3 wt % of CNF or the derivative thereof. In some embodiments, the composition comprises between about 0.01 wt % to about 2 wt % of CNF or the derivative thereof. In some embodiments, the composition comprises between about 0.01 wt % to about 1 wt % of CNF or the derivative thereof. For example, in some embodiments, the composition comprises between about 0.06 wt % to about 3 wt % of CNF or the derivative thereof.

In some embodiments, the composition comprises CNF or the derivative thereof and the adhering agent in a molar ratio of about 100:1. In some embodiments, the molar ratio of CNF or the derivative thereof to the adhering agent is about 90:1. In some embodiments, the molar ratio of CNF or the derivative thereof to the adhering agent is about 80:1. In some embodiments, the molar ratio of CNF or the derivative thereof to the adhering agent is about 70:1. In some embodiments, the molar ratio of CNF or the derivative thereof to the adhering agent is about 60:1. In some embodiments, the molar ratio of CNF or the derivative thereof to the adhering agent is about 50:1. In some embodiments, the molar ratio of CNF or the derivative thereof to the adhering agent is about 40:1. In some embodiments, the molar ratio of CNF or the derivative thereof to the adhering agent is about 30:1. In some embodiments, the molar ratio of CNF or the derivative thereof to the adhering agent is about 20:1. In some embodiments, the molar ratio of CNF or the derivative thereof to the adhering agent is about 10:1. In some embodiments, the molar ratio of CNF or the derivative thereof to the adhering agent is about 5:1. In some embodiments, the molar ratio of CNF or the derivative thereof to the adhering agent is about 2:1. In some embodiments, the molar ratio of CNF or the derivative thereof to the adhering agent is about 1:1. In some embodiments, the molar ratio of CNF or the derivative thereof to the adhering agent is about 1:100. In some embodiments, the molar ratio of CNF or the derivative thereof to the adhering agent is about 1:90. In some embodiments, the molar ratio of CNF or the derivative thereof to the adhering agent is about 1:80. In some embodiments, the molar ratio of CNF or the derivative thereof to the adhering agent is about 1:70. In some embodiments, the molar ratio of CNF or the derivative thereof to the adhering agent is about 1:60. In some embodiments, the molar ratio of CNF or the derivative thereof to the adhering agent is about 1:50. In some embodiments, the molar ratio of CNF or the derivative thereof to the adhering agent is about 1:40. In some embodiments, the molar ratio of CNF or the derivative thereof to the adhering agent is about 1:30. In some embodiments, the molar ratio of CNF or the derivative thereof to the adhering agent is about 1:20. In some embodiments, the molar ratio of CNF or the derivative thereof to the adhering agent is about 1:10. In some embodiments, the molar ratio of CNF or the derivative thereof to the adhering agent is about 1:5. In some embodiments, the molar ratio of CNF or the derivative thereof to the adhering agent is about 1:2.

In some embodiments, the composition comprises between about 0.01 wt % to about 50 wt % of the adhering agent. In some embodiments, the composition comprises between about 0.01 wt % to about 40 wt % of the adhering agent. In some embodiments, the composition comprises between about 0.01 wt % to about 30 wt % of the adhering agent. In some embodiments, the composition comprises between about 0.01 wt % to about 20 wt % of the adhering agent. In some embodiments, the composition comprises between about 0.01 wt % to about 10 wt % of the adhering agent. In some embodiments, the composition comprises between about 0.01 wt % to about 9 wt % of the adhering agent. In some embodiments, the composition comprises between about 0.01 wt % to about 8 wt % of the adhering agent. In some embodiments, the composition comprises between about 0.01 wt % to about 7 wt % of the adhering agent. In some embodiments, the composition comprises between about 0.01 wt % to about 6 wt % of the adhering agent. In some embodiments, the composition comprises between about 0.01 wt % to about 5 wt % of the adhering agent. In some embodiments, the composition comprises between about 0.01 wt % to about 4 wt % of the adhering agent. In some embodiments, the composition comprises between about 0.01 wt % to about 3 wt % of the adhering agent. In some embodiments, the composition comprises between about 0.01 wt % to about 2 wt % of the adhering agent. In some embodiments, the composition comprises between about 0.01 wt % to about 1 wt % of the adhering agent. For example, in some embodiments, the composition comprises between about 0.07 wt % to about 9 wt % of the adhering agent.

In various embodiments, the at least one therapeutic agent comprises chitosan (CS) or a derivative thereof. Thus, in various embodiments, the composition comprises CS or a derivative thereof.

In one embodiment, the CS or a derivative thereof is a long chain polymer. In one embodiment, the CS or a derivative thereof is a short chain polymer. In one embodiment, the CS or a derivative thereof permeates the composition comprising CNF or a derivative thereof. In one embodiment, the CS or a derivative thereof is suspended in aqueous solution. In one embodiment, the CS or a derivative thereof is modified into a nanoparticle.

In one embodiment, the composition is a homogenous composition. In one embodiment, the composition is a heterogeneous composition.

In one embodiment, the composition is an emulsion. In one embodiment, the composition is a dispersion.

In some embodiments, the composition comprises between about 0.01 wt % to about 50 wt % of CS or the derivative thereof. In some embodiments, the composition comprises between about 0.01 wt % to about 40 wt % of CS or the derivative thereof. In some embodiments, the composition comprises between about 0.01 wt % to about 30 wt % of CS or the derivative thereof. In some embodiments, the composition comprises between about 0.01 wt % to about 20 wt % of CS or the derivative thereof. In some embodiments, the composition comprises between about 0.01 wt % to about 10 wt % of CS or the derivative thereof. In some embodiments, the composition comprises between about 0.01 wt % to about 9 wt % of CS or the derivative thereof. In some embodiments, the composition comprises between about 0.01 wt % to about 8 wt % of CS or the derivative thereof. In some embodiments, the composition comprises between about 0.01 wt % to about 7 wt % of CS or the derivative thereof. In some embodiments, the composition comprises between about 0.01 wt % to about 6 wt % of CS or the derivative thereof. In some embodiments, the composition comprises between about 0.01 wt % to about 5 wt % of CS or the derivative thereof. In some embodiments, the composition comprises between about 0.01 wt % to about 4 wt % of CS or the derivative thereof. In some embodiments, the composition comprises between about 0.01 wt % to about 3 wt % of CS or the derivative thereof. In some embodiments, the composition comprises between about 0.01 wt % to about 2 wt % of CS or the derivative thereof. In some embodiments, the composition comprises between about 0.01 wt % to about 1 wt % of CS or the derivative thereof. For example, in some embodiments, the composition comprises between about 0.06 wt % to about 3 wt % of CS or the derivative thereof.

In some embodiments, the composition comprises CS or the derivative thereof and CNF or the derivative thereof in a molar ratio of about 100:1. In some embodiments, the molar ratio of CS or the derivative thereof to CNF or the derivative thereof is about 90:1. In some embodiments, the molar ratio of CS or the derivative thereof to CNF or the derivative thereof is about 80:1. In some embodiments, the molar ratio of CS or the derivative thereof to CNF or the derivative thereof is about 70:1. In some embodiments, the molar ratio of CS or the derivative thereof to CNF or the derivative thereof is about 60:1. In some embodiments, the molar ratio of CS or the derivative thereof to CNF or the derivative thereof is about 50:1. In some embodiments, the molar ratio of CS or the derivative thereof to CNF or the derivative thereof is about 40:1. In some embodiments, the molar ratio of CS or the derivative thereof to CNF or the derivative thereof is about 30:1. In some embodiments, the molar ratio of CS or the derivative thereof to CNF or the derivative thereof is about 20:1. In some embodiments, the molar ratio of CS or the derivative thereof to CNF or the derivative thereof is about 10:1. In some embodiments, the molar ratio of CS or the derivative thereof to CNF or the derivative thereof is about 5:1. In some embodiments, the molar ratio of CS or the derivative thereof to CNF or the derivative thereof is about 2:1. In some embodiments, the molar ratio of CS or the derivative thereof to CNF or the derivative thereof is about 1:1. In some embodiments, the molar ratio of CS or the derivative thereof to CNF or the derivative thereof is about 1:100. In some embodiments, the molar ratio of CS or the derivative thereof to CNF or the derivative thereof is about 1:90. In some embodiments, the molar ratio of CS or the derivative thereof to CNF or the derivative thereof is about 1:80. In some embodiments, the molar ratio of CS or the derivative thereof to CNF or the derivative thereof is about 1:70. In some embodiments, the molar ratio of CS or the derivative thereof to CNF or the derivative thereof is about 1:60. In some embodiments, the molar ratio of CS or the derivative thereof to CNF or the derivative thereof is about 1:50. In some embodiments, the molar ratio of CS or the derivative thereof to CNF or the derivative thereof is about 1:40. In some embodiments, the molar ratio of CS or the derivative thereof to CNF or the derivative thereof is about 1:30. In some embodiments, the molar ratio of CS or the derivative thereof to CNF or the derivative thereof is about 1:20. In some embodiments, the molar ratio of CS or the derivative thereof to CNF or the derivative thereof is about 1:10. In some embodiments, the molar ratio of CS or the derivative thereof to CNF or the derivative thereof is about 1:5. In some embodiments, the molar ratio of CS or the derivative thereof to CNF or the derivative thereof is about 1:2.

In some embodiments, the adhesive agent comprises at least one polymer.

In some embodiments, the polymer has molecular weight of between about 5 kDa to about 100,000 kDa. For example, in some embodiments, the polymer has a molecular weight of between about between about 10 kDa to about 100,000 kDa, 800 kDa to about 90,000 kDa between about 800 kDa to about 3,000 kDa, between about 5 kDa to about 2,000 kDa, between about 5 kDa to about 1,500 kDa, between about 5 kDa to about 1,000 kDa, between about 5 kDa to about 800 kDa, between about 5 kDa to about 500 kDa, between about 5 kDa to about 300 kDa, between about 5 kDa to about 200 kDa, between about 5 kDa to about 130 kDa, or between about 80 kDa to about 130 kDa.

In some embodiments, the polymer is a water-soluble polymer, biodegradable polymer, biocompatible polymer, or any combination thereof.

In other embodiments, the polymer is a water-resistant polymer.

In some embodiments, the polymer is a hydrophilic polymer. In other embodiments, the polymer is a hydrophobic polymer.

In some embodiments, the polymer is a cationic polymer, anionic polymer, neutral polymer, or any combination thereof.

The polymer may be a straight chain polymer (i.e., linear polymer) or a branched chain polymer (i.e., branched polymer), including hyperbranched polymers. In some embodiments, the polymer is cross-linked.

In some embodiments, the polymer is a homopolymer. In other embodiments, the polymer a copolymer. In some embodiments, the polymer a block copolymer that is a diblock, triblock, tetrablock, pentablock, or at least six block copolymer.

In some embodiments, the polymer is a polyhydroxy polymer.

Examples of such polymers include, but are not limited to, a polyvinyl alcohol (PVA), polyethylene glycol (PEG), 1,2-distearoyl-sn-glycero-3-phosphoethanolamine (DSPE), 1,2-distearoyl-sn-glycero-3-phosphoethanolamine-polyethylene glycol (DSPE-PEG), 1,2-distearoyl-sn-glycero-3-phosphoethanolamine-N-[amino (polyethylene glycol)-2000](DSPE-PEG (2000)-amine), polyethyleneimine (PEI), poly (lactide-co-glycolic acid) (PLGA), biodegradable PLGA, poly (lactide-co-glycolic acid)-polyethylene glycol (PLGA-PEG), biodegradable PLGA-PEG, poly (lactide-co-glycolic acid)-block-polyethylene glycol (PLGA-b-PEG), biodegradable PLGA-b-PEG, poly (ethylene oxide) (PEO), PEO block copolymer, poly (ethylethylene) (PEE), poly (butadiene) (PB or PBD), poly (styrene) (PS), poly (isoprene) (PI), polyanhydride, polyanhydride-block-PEG copolymers, zwitterionic poly (carbobetaine), zwitterionic poly (sulfobetaine)-containing, zwitterionic poly (carbobetaine) and zwitterionic poly (sulfobetaine)-containing copolymers, poly (acrylic acid-co-distearin acrylate), poly (trimethylene carbonate)-block-poly (L-gluatamic acid), poly (ethylene glycol-block-L-aspartic acid), poly (2-hydroxyethyl-co-octadecyl aspartamide), poly (ethylene glycol-co-trimethylene carbonate-co-caprolactone, polypropylene oxide block copolymers, polyethylene oxide-block-polypropylene oxide copolymers, poly (F-caprolactone) (PCL), PCL diblock co-polymer, poly (ethylene oxide)-block-poly (F-caprolactone) (PEO-b-PCL) based diblock copolymers, poly (lactic acid), poly (glycolide), poly (lactic-coglycolic acid), poly (3-hydroxybutyrate), polyamine, polyalkyleneimine (e.g., polyethyleneimine), polyallylamine, polyamidoamine, poly (amino-co-ester), chitosan, poly (2-N,N-dimethylaminoethylmethacrylate), poly-L-lysine, maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid, PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols, PEG-c-DOMG, PEG-c-DMA, PEG-s-DMG, polyethylene glycol-lipid is N-[(methoxy poly (ethylene glycol)2000)carbamyl]-1,2-dimyristyloxlpropyl-3-amine (PEG-c-DMA), polyethylene glycol-lipid is PEG-c-DOMG), pegylated diacylglycerol (PEG-DAG), 1-(monomethoxy-polyethyleneglycol)-2,3-dimyristoylglycerol (PEG-DMG), pegylated phosphatidylethanoloamine (PEG-PE), PEG succinate diacylglycerol (PEG-S-DAG) such as 4-O-(2′,3′-di (tetradecanoyloxy)propyl-1-O-(w-methoxy (polyethoxy)ethyl)butanedioate (PEG-S-DMG), pegylated ceramide (PEG-cer), PEG dialkoxypropylcarbamate, 1,2-distearoyl-sn-glycero-3-phosphoethanolamine PEG maleimide (DSPE-PEG-maleimide), DSPE-PEG-carboxy-N-hydroxysuccinimide (NHS), DSPE-PEG-folate, DSPE-PEG-biotin, maltrin, an aminoalkyl methacrylate copolymer available under the trade name of EUDRAGIT® (type E100 or EPO), polyvinylacetal diethylaminoacetate e.g., AEA® available from Sankyo Company Limited, Tokyo (Japan), carboxymethylethylcellulose, cellulose acetate phthalate (CAP), cellulose acetate succinate, methylcellulose phthalate, hydroxymethylethylcellulose phthalate, hydroxypropylmethylcellulose phthalate (HPMCP), hydroxypropylmethylcellulose acetate succinate (HPMCAS), polyvinyl alcohol phthalate, polyvinyl butyrate phthalate, polyvinyl acetal phthalate (PVAP), a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate, a copolymer of methacrylic acid/methyl methacrylate, cellulose acetate hexahydrophthalate, hydroxypropyl methylcellulose hexahydrophthalate, hydroxypropyl methylcellulose phthalate, cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), methacrylic acid-methyl methacrylate copolymer, ethyl methacrylate-methylmethacrylate-chlorotrimethylammonium ethyl methacrylate copolymer, or any combination thereof. For example, in some embodiments, the polymer is PVA.

Examples of water-soluble polymers include, but are not limited to, homopolymers and copolymers of N-vinyl lactams, including homopolymers and copolymers of N-vinyl pyrrolidone, e.g. polyvinylpyrrolidone (PVP), copolymers of N-vinyl pyrrolidone and vinyl acetate or vinyl propionate, PVA, cellulose esters and cellulose ethers, in particular methylcellulose and ethylcellulose, hydroxyalkylcelluloses, in particular hydroxypropylcellulose, hydroxyalkylalkylcelluloses, and hydroxypropylmethylcellulose, cellulose phthalates, succinates, butyrates, or trimellitates, in particular cellulose acetate phthalate, hydroxypropylmethylcellulose phthalate, hydroxypropylmethylcellulose succinate, and hydroxypropylmethylcellulose acetate succinate; high molecular polyalkylene oxides such as polyethylene oxide and polypropylene oxide and copolymers of ethylene oxide and propylene oxide, polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly (hydroxyalkyl acrylates), poly (hydroxyalkyl methacrylates), polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol, polyethylene glycol oligo- and polysaccharides such as carrageenans, galactomannans xanthan gum, and corn starch, or mixtures of one or more thereof.

Examples of hydrophilic polymers include, but are not limited to, hydroxypropyl celluloses (HPC), hydroxypropyl methylcelluloses, methylcelluloses, polyethylene oxides, sodium carboxymethyl celluloses, and the like, or combinations thereof.

Examples of water-insoluble polymers include, but are not limited to, acrylic polymers, methacrylic acid polymers, acrylic copolymers, such as a methacrylic acid-ethyl acrylate copolymer available under the trade name of EUDRAGIT® (type L, RL, RS and NE30D), and their respective esters, zein, waxes, shellac and hydrogenated vegetable oil, cellulose derivatives, such as ethyl cellulose, cellulose acetate, cellulose acetate butyrate, and the like.

In some embodiments, the polymer comprises one or more functional groups. In some embodiments, the functional group is an azide functional group, maleimide functional group, carboxyl functional group, amine functional group, hydrazine functional group, dibenzo-cyclooctyne functional group, or any combination thereof.

In some embodiments, the polymer stabilizes the composition.

In some embodiments, the adhering agent specifically binds CNF to a surface of a material of interest. In some embodiments, the material of interest is selected from a textile material, paper, glass, metal, wood, plastic, or any combination thereof.

Examples of textile material include, but are not limited to textile materials comprising polyester, polypropylene, polyethylene, polyurethane, polyacrylonitrile, cotton, linen, hemp, nylon, silk, rayon, modal, cellulose, denim, wool, chenille, leather, spandex, or any combination or any blend thereof.

In some embodiments, the composition further comprises at least one therapeutic agent.

In some embodiments, the adhering agent specifically binds CNF and the therapeutic agent to a surface of a material of interest. In some embodiments, the material of interest is any material described herein.

In some embodiments, the composition comprises between about 0.01 wt % to about 50 wt % of the therapeutic agent. In some embodiments, the composition comprises between about 0.01 wt % to about 40 wt % of the therapeutic agent. In some embodiments, the composition comprises between about 0.01 wt % to about 30 wt % of the therapeutic agent. In some embodiments, the composition comprises between about 0.01 wt % to about 20 wt % of the therapeutic agent. In some embodiments, the composition comprises between about 0.01 wt % to about 10 wt % of the therapeutic agent. In some embodiments, the composition comprises between about 0.01 wt % to about 9 wt % of the therapeutic agent. In some embodiments, the composition comprises between about 0.01 wt % to about 8 wt % of the therapeutic agent. In some embodiments, the composition comprises between about 0.01 wt % to about 7 wt % of the therapeutic agent. In some embodiments, the composition comprises between about 0.01 wt % to about 6 wt % of the therapeutic agent. In some embodiments, the composition comprises between about 0.01 wt % to about 5 wt % of the therapeutic agent. In some embodiments, the composition comprises between about 0.01 wt % to about 4 wt % of the therapeutic agent. In some embodiments, the composition comprises between about 0.01 wt % to about 3 wt % of the therapeutic agent. In some embodiments, the composition comprises between about 0.01 wt % to about 2 wt % of the therapeutic agent. In some embodiments, the composition comprises between about 0.01 wt % to about 1 wt % of the therapeutic agent. For example, in some embodiments, the composition comprises between about 0.01 wt % to about 2 wt % of the therapeutic agent.

In some embodiments, the therapeutic agent is a hydrophilic therapeutic agent. In some embodiments, the therapeutic agent is a hydrophobic therapeutic agent.

In some embodiments, the therapeutic agent is an antimicrobial agent. In some embodiments, the antimicrobial agents comprises an antibacterial agent, antibiotic, antiviral agent, antifungal agent, or any combination thereof.

In some embodiments, the antimicrobial agent may be a prodrug form of an antimicrobial agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an antimicrobial agent in the compositions, among other things, may increase the concentration of the antimicrobial agent in the compositions of the present disclosure. In certain embodiments, an antimicrobial agent may be chemically modified with an alkyl or acyl group or some form of lipid. The selection of such a chemical modification, including the substituent (s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

Examples of antibiotic agents include, but are not limited to: levofloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

Examples of anti-viral agents include, but are not limited to proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. Many examples of antiviral compounds that can be used in combination with the compositions of the invention are known in the art and include but are not limited to: nirmatrelvir, ritonavir, paxlovid, rifampicin, nucleoside reverse transcriptase inhibitors (e.g., AZT, ddI, ddC, 3TC, d4T), non-nucleoside reverse transcriptase inhibitors (e.g., Efavirenz, Nevirapine), protease inhibitors (e.g., aprenavir, indinavir, ritonavir, and saquinavir), idoxuridine, cidofovir, acyclovir, ganciclovir, zanainivir, amantadine, and Palivizumab. Other examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime, zinc, heparin, anionic polymers.

Examples of antifungal agents include, but are not limited to: polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate, Tolindate; Tolnaftate; Triacetin; Triafuigin; Undecylenic Acid; Viridoflilvin; Zinc Undecylenate; and Zinoconazole Hydrochloride. Antifungal agents and their dosages, routes of administration and recommended usage are known in the art and have been described in such literature as the Physician's Desk Reference (60th ed., 2006).

Examples of other therapeutic agents useful in the present invention include, but are not limited to, one or more drugs, proteins, amino acids, peptides, antibodies, antibiotics, small molecules, RNA molecules, siRNA molecules, DNA molecules, therapeutic moieties, antiproliferative agents, antineoplastic agents, vitamins, minerals, lipids, saccharides, metals, amino acids (and precursors), nucleic acids and precursors, or any combinations thereof. Examples of antiproliferative agents include compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

In some embodiments, the composition further comprises a carrier.

In one embodiment, the carrier comprises an aqueous solution. In some embodiments, the aqueous solution is an acidic solution, basic solution, or neutral solution. For example, in some embodiments, the aqueous solution comprises water, methanol, ethanol, propanol, isopropyl alcohol, butyl alcohol, diethyl ether, acetone, methyl acetate, ethyl acetate, acetic acid, or any combination thereof.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. In many cases, it will be preferable to include isotonic agents, for example, sugars, sodium chloride, or polyalcohols such as mannitol and sorbitol, in the composition.

In one embodiment, suitable carriers include, but are not limited to, diluents and sterile aqueous or organic solutions. Carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate. Aqueous carriers suitable for use in the present application include, but are not limited to, water, ethanol, alcoholic/aqueous solutions, glycerol, emulsions or suspensions, including saline and buffered media.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. Liquid carriers suitable for use in the present application include, but are not limited to, water (partially containing additives, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Diluents may be added to the formulations described herein. In various embodiments, diluents include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc, and/or mixtures of any of the foregoing. Specific examples of: microcrystalline cellulose include those sold under the Trademark Avicel (FMC Corp., Philadelphia, Pa.), for example, Avicel™ pH101, Avicel™ pH102 and Avicel™ pH112; lactose include lactose monohydrate, lactose anhydrous and Pharmatose DCL21; dibasic calcium phosphate includes Emcompress.

In various aspects, the composition further comprises one or more stabilizers. In some embodiments, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, corn starch, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly (vinylchloride), micro-porous polyamides, microporous modacrylic copolymers, microporous styrene-acrylic and its copolymers); porous polysulfones, halogenated poly (vinylidene), polychloroethers, acetal polymers, polyesters prepared by esterification of a dicarboxylic acid or anhydride with an alkylene polyol, poly (alkylenesulfides), phenolics, polyesters, asymmetric porous polymers, cross-linked olefin polymers, hydrophilic microporous homopolymers, copolymers or interpolymers having a reduced bulk density, and other similar materials, poly (urethane), cross-linked chain-extended poly (urethane), poly (imides), poly (benzimidazoles), collodion, regenerated proteins, semi-solid cross-linked poly (vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

In various aspects, the composition further comprises one or more additional ingredient. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; antiseptics; antiviral agents; anticoagulants; stabilizing agents; and pharmaceutically acceptable polymeric or hydrophobic materials. Other “additional ingredients” which may be included in the compositions of the disclosure are known in the art and described, for example in Genaro, ed. (1985, Remington's Pharmaceutical Sciences, Mack Publishing Co., Easton, PA), which is incorporated herein by reference.

In some embodiments, the composition is a biodegradable composition. In some embodiments, the composition is a medical biodegradable composition.

In some embodiments, the composition is a biocompatible composition. In some embodiments, the composition is a medical biocompatible composition.

In some embodiments, the composition is a pharmaceutical composition. In some embodiments, the composition further comprises at least one pharmaceutically acceptable carrier.

In various aspects, the composition of the present invention increases barrier properties against at least one particle of interest.

In some embodiments, the particle of interest has a particle size (e.g., average hydrodynamic diameter of the particle) of about 0.1 nm to about 100,000 nm. In some embodiments, the particle of interest has a particle size (e.g., average hydrodynamic diameter of the particle) of about 0.1 nm to about 1,000 nm. In some embodiments, the particle of interest has a particle size (e.g., average hydrodynamic diameter of the particle) of about 1 nm to about 100,000 nm. In some embodiments, the particle of interest has a particle size (e.g., average hydrodynamic diameter of the particle) of about 1 nm to about 50,000 nm. In some embodiments, the particle of interest has a particle size (e.g., average hydrodynamic diameter of the particle) of about 1 nm to about 10,000 nm. In some embodiments, the particle of interest has a particle size (e.g., average hydrodynamic diameter of the particle) of about 1 nm to about 1,000 nm. In some embodiments, the particle of interest has a particle size (e.g., average hydrodynamic diameter of the particle) of about 10 nm to about 100 nm. In some embodiments, the particle of interest has a particle size (i.e., average hydrodynamic diameter of the composition) of between about 50 nm to about 250 nm. In some embodiments, the particle of interest has a particle size (i.e., average hydrodynamic diameter of the composition) of between about 50 nm to about 200 nm. In some embodiments, the particle of interest has a particle size (i.e., average hydrodynamic diameter of the composition) of between about 100 nm to about 200 nm. In some embodiments, the particle of interest has a particle size (i.e., average hydrodynamic diameter of the composition) of about 100 nm to about 160 nm. In some embodiments, the particle of interest has a particle size (e.g., average hydrodynamic diameter of the particle) of about 100 nm to about 150 nm. In some embodiments, the particle of interest has a particle size (e.g., average hydrodynamic diameter of the particle) of about 200 nm to about 400 nm. In some embodiments, the particle of interest has a particle size (e.g., average hydrodynamic diameter of the particle) of about 40 nm to about 60 nm. For example, in various embodiments, the particle of interest has an average particle size (e.g., average hydrodynamic diameter of the composition) below about 250 nm.

In some embodiments, the particle of interest is an aerosol particle. Examples of such aerosol particles include, but are not limited to, aerosol particles comprising at least one aerosol pathogen (e.g., aerosol microorganism, such as aerosol bacterium, aerosol virus, aerosol fungus, aerosol parasite, etc.), aerosol pollutant, aerosol toxin, aerosol allergen,aerosol particle associated with a disease or disorder, or any combination thereof.

In some embodiments, the particle of interest is an airborne allergen. In certain embodiments, the allergen is a seasonal allergen. Examples of such airborne allergens include, but are not limited to, pollen allergens (tree, weed, and grass pollen allergens), mite allergens (from e.g. house dust mites and storage mites), insect allergens (e.g. inhalant origin allergens), animal allergens (from e.g. hair, dander feathers from e.g. dog, cat, horse, rat, mouse, guinea pig, rabbit, bird), fungal or mold allergens.

Examples of pollen allergens include, but are not limited to, tree pollen, including pollen from trees belonging to the orders Fagales, Lamiales, Proteales, Pinales, Fabales, Malpighiales, Sapindales, Myrtales, Rosales and Arecales; weed pollen, including pollen from weeds belonging to the families Asteraceae, Amaranthaceae, Plantaginaceae, Uritaceae and Euphorbiaceae; grass pollen, including pollen from grass belonging to the genera Oryza, Phragmites, Cynodon, Paspalum, Sorghum, Zea, Dactylis, Festuca, Lolium, Poa, Anthoxanthym, Avena, Holcus, Phalaris, Agrostis, Alopecurus, Phleum, Bromus, Hordeum, Secale, and Triticum; microbial species, e.g. bacteria and fungi, that colonize pollen; and pollen-derived submicronic and paucimicronic particles.

In some embodiments, the composition breaks a membrane of at least one cell of interest. Thus, in some embodiments, the composition modulates cell lysis of at least one cell of interest. In some embodiments, the composition induces cell lysis of at least one cell of interest.

In some embodiments, the composition results in induced cell lysis of at least one cell of interest that is increased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the composition results in induced cell lysis of at least one cell of interest that is at least about 0.01 fold higher than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the comparator is a cell not exposed to the composition of the present invention.

In some embodiments, the cell of interest is a pathogen cell. In some embodiments, the pathogen cell is a microorganism cell.

In some embodiments, the microorganism is a bacterium, virus, fungus, parasite, and any combination thereof. In one embodiment, the microorganism is resistant to at least one antibiotic.

Examples of such microorganisms include, but are not limited to bacteria, such as Acinetobacter baumannii, Actinomyces israelii, Bacillus anthracis, Bacillus cereus, Bacillus subtilis, Bacteroides fragilis, Bartonella henselae, Bartonella Quintana, Bordetella pertussis, Borrelia burgdorferi, Borrelia garinii, Borrelia afzelii, Borrelia recurrentis, Brevundimonas diminuta, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Ehrlichia canis, Ehrlichia chaffeensis, Enterobacter cloacae, Enterococcus faecalis, Enterococcus faecium, Escherichia coli, Francisella tularensis, Haemophilus influenzae, Helicobacter pylori, Klebsiella pneumoniae, Lactobacillus rhamnosus, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Proteus mirabills, Pseudomonas aeruginosa, Nocardia asteroids, Rickettsia rickettsia, Salmonella enterica, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Candida species, such as Candida albicans, Candida tropicalis, Candida glabrata, Candida parapsilosis, Candida krusei, Candida lusitaniae, Candida kefyr, Candida guilliermondii, and Candida dubliniensis, Yersinia pestis, Yersinia enterocolitica, Yersinia pseudotuberculosis, and any combination thereof.

Examples of virus include, but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picomaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of RNA viruses that are antigens in vertebrate animals include, but are not limited to, the following: members of the family Retroviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picomaviridae, including the genus Enterovirus, poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus; Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever, virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type I, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, (Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric coronavirus (SARS-CoV, MERS-CoV, SARS-CoV-2), and Feline infectious peritonitis (Feline coronavirus); the family Fiersviridae, including MS2 coliphage.

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc).

In some embodiments, the composition decreases a contact angle between the at least one particle and the material of interest.

In some embodiments, the composition results in decreased contact angle between the at least one particle and the material of interest that is decreased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the composition results in decreased contact angle between the at least one particle and the material of interest that is at least about 0.01 fold lower than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or less, including all values and ranges in-between.

In some embodiments, the comparator is a material not exposed to the composition of the present invention.

In some embodiments, the composition decreases a size of at least one pore of the material of interest.

In some embodiments, the composition results in decreased size of at least one pore of the material of interest that is decreased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the composition results in size of at least one pore of the material of interest that is at least about 0.01 fold lower than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or less, including all values and ranges in-between.

In some embodiments, the comparator is a material not exposed to the composition of the present invention.

### Devices

In one aspect, the present invention provides a device comprising the composition of the present invention. In some embodiments, the device comprises the composition of the present invention and a material of interest. In some embodiments, the device comprises at least one layer of the composition of the present invention coated on the surface of the material of interest. The material of interest is any material described herein (e.g., paper, glass, metal, wood, plastic, and/or textile material, such as polyester, polypropylene, polyethylene, polyurethane, polyacrylonitrile, cotton, linen, nylon, silk, rayon, modal, cellulose, denim, wool, chenille, leather, and/or spandex).

In some embodiments, the device is a mask, clothing, fabric, textile, furniture, carpet, curtain, upholstery, filter, or the like. In one embodiment, the filter is an air filter. In one embodiment, the mask is one-layered. In one embodiment, the mask is multi-layered.

In some embodiments, the device has an increased filtration efficiency.

In some embodiments, the device increases barrier properties against at least one particle of interest. The particle of interest is any particle described herein (e.g., aerosol particle).

In some embodiments, the device breaks a membrane of at least one cell of interest. Thus, in some embodiments, the device modulated the cell lysis of at least one cell of interest. In some embodiments, the device induces cell lysis of at least one cell of interest. The at least one cell of interest is any cell described herein (e.g., pathogen cell).

In some embodiments, the device results in induced cell lysis of at least one cell of interest that is increased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the device results in induced cell lysis of at least one cell of interest that is at least about 0.01 fold higher than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the comparator is a cell not exposed to the composition of the present invention.

### Methods of Use

In one aspect, the present invention provides a method of increasing filtration efficiency a material of interest. In another aspect, the present invention provides a method of increasing barrier properties of a material of interest against at least one particle. In another aspect, the present invention provides a method of applying at least one composition of the present invention to a material of interest.

The material of interest is any material described herein (e.g., paper, glass, metal, wood, plastic, and/or textile material, such as polyester, polypropylene, polyethylene, polyurethane, polyacrylonitrile, cotton, linen, nylon, silk, rayon, modal, cellulose, denim, wool, chenille, leather, and/or spandex).

The particle of interest is any particle described herein (e.g., aerosol particle).

In some embodiments, the method comprises applying at least one layer of the composition to the surface of the material of interest.

In some embodiments, the at least one layer of the composition is applied to the surface of the material of interest by spraying, painting, brushing, soaking, or any combination thereof.

In some embodiments, the at least one layer of the composition is applied to the surface of the material of interest by spraying. In some embodiments, the composition is sprayed to the surface of the material of interest at least 1 time. In some embodiments, the composition is sprayed to the surface of the material of interest at least 2 times. In some embodiments, the composition is sprayed to the surface of the material of interest at least 3 times. In some embodiments, the composition is sprayed to the surface of the material of interest at least 4 times. In some embodiments, the composition is sprayed to the surface of the material of interest at least 5 times. In some embodiments, the composition is sprayed to the surface of the material of interest at least 6 times. In some embodiments, the composition is sprayed to the surface of the material of interest at least 7 times. In some embodiments, the composition is sprayed to the surface of the material of interest at least 8 times. In some embodiments, the composition is sprayed to the surface of the material of interest at least 9 times. In some embodiments, the composition is sprayed to the surface of the material of interest at least 10 times.

In one aspect, the present invention provides a method of breaking a membrane of at least one cell of interest.

In another aspect, the present invention provides a method of modulating cell lysis of at least one cell of interest.

In another aspect, the present invention provides a method of killing a cell of interest or inhibiting the grown of a cell of interest.

In some embodiments, the method comprises contacting the at least one cell of interest with the composition or device of the present invention. In some embodiments, the composition or the device induces cell lysis of at least one cell of interest.

The at least one cell of interest is any cell described herein (e.g., pathogen cell).

In one aspect, the present invention provides a method for reducing the number of particles (e.g., pathogen cells, microorganisms, etc.) attached to the surface of a medical device or the surface of a subject's body (e.g., the skin of the subject, or a mucous membrane of the subject, such as the throat).

In some embodiments, the method comprises using the composition of the present invention to coat the surface of a medical device, thus inhibiting or disrupting pathogenic or microbial growth and/or inhibiting or disrupting the formation of biofilm on the surface of the medical device. The compositions of the invention find further use in preventing or reducing the growth or proliferation of pathogens or microorganisms and/or biofilm-embedded pathogens or microorganisms on the surface of a medical device or on the surface of a subject's body. However, the invention is not limited to applications in the medical field. Rather, the invention includes using the compositions described herein as an antimicrobial and/or antibiofilm agent in any setting.

In another aspect, the invention provides a method for preventing a disease or disorder related to the detrimental growth and/or proliferation of a pathogenic cell in vivo, ex vivo or in vitro.

In one aspect, the present invention provides a method of preventing a disease or disorder associated with at least one aerosol particle in a subject. In some embodiments, the method comprises the subject wearing the device of the present invention over the subject's mouth and nose.

In one aspect, the present invention provides a method of preventing a disease or disorder associated with at least one aerosol particle. In some embodiments, the method comprises the device of the present invention. In some embodiments, the device is an air filter in an air purifier that reduces the level of the aerosol particles associated with the disease or disorder.

In some embodiments, the at least one aerosol particle is any aerosol particle described herein (e.g., aerosol microorganism, such as an aerosol bacterium, aerosol virus, aerosol pathogen, or aerosol fungus, aerosol pollutant, aerosol toxin, and/or aerosol particle associated with a disease or disorder).

In various embodiments, the disease or disorder associated with at least one aerosol particle is a disease or disorder associated with at least one microorganism. In various embodiments, the disease or disorder associated with at least one aerosol particle is an infection.

In some embodiments, the infection is a bacterial infection, viral infection, fungal infection, parasitic infection, or any combination thereof.

In some embodiments, the method prevents a viral infection. In some embodiments, the viral infection is an infection caused by any virus described herein. Examples of such viral infection include, but are not limited to, coronavirus disease, such as COVID-19 or flu.

In some embodiments, the method prevents a bacterial infection. In some embodiments, the bacterial infection is an infection caused by any bacterium described herein. For example, in one embodiment, the method prevents a gram-positive bacterial infection. In one embodiment, the bacterial infection is resistant to antibiotics. For example, in one embodiment, the bacterial infection is resistant to one or more of beta-lactams, including methicillin, oxacillin, or penicillin, tetracyclines, gentamicin, kanamycin, erythromycin, spectinomycin, and vancomycin.

In some embodiments, the method prevents a fungal infection. In some embodiments, the fungal infection is an infection caused by any fungus described herein.

In some embodiments, the method prevents a parasitic infection. In some embodiments, the parasitic infection is an infection caused by any parasite described herein.

In some embodiments, the method prevents an allergic reaction. In some embodiments, the allergic reaction is caused by any allergen described herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Cellulose Nanofibers (CNF) as Mask/Personal Protective Equipment (PPE) Surface Agents for Enhanced Anti-Bacterial Performance

The utilization of face-covering masks as an extended form of PPE has led to exponential waste measures during the Covid-19 pandemic, with estimations of up to 7,200 tons of medical-type waste daily. A primary cause of this waste are layered, disposable surgical masks that are constructed by melt-blown nonwovens usually made from non-biodegradable thermoplastic polymers, such as polypropylene. To increase widespread sustainable options to the public, commercialized or DIY-based fabric masks serve as a solution, but their resistance to harmful molecules is less than the medical-grade masks due to their structure of fabrics, leaving space for penetration.

The demand for sustainable and secure solutions to mitigate aerosol particle transmission has seen a considerable increase in various directions. For instance, a previous study demonstrated an effective avenue utilizing electrospun cellulose nanofiber/poly-vinyl alcohol (CNF/PVA) biodegradable nanofibrous air filters to combat air particulate matter transmission (Zhang et al., 2020, Chemical Engineering Journal, 399:125768). In a different approach, other studies employed of nanocellulose for bioavailable in vitro drug release systems by integrating curcumin-loaded cellulose nanocrystals in a PVA medium (Gunathilake et al; 2022, Cellulose (Lond); 29:1821-1840).

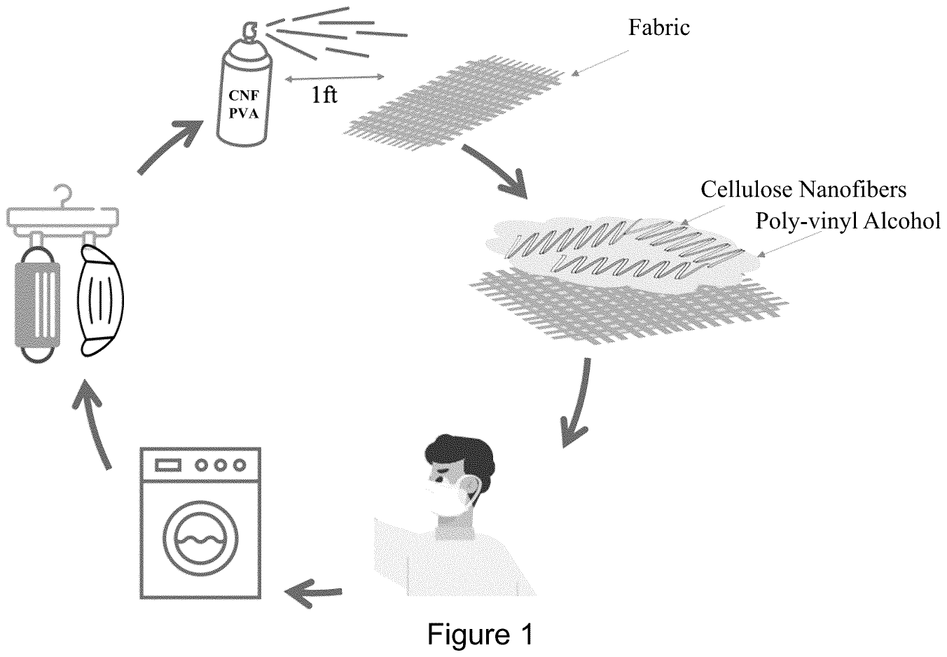

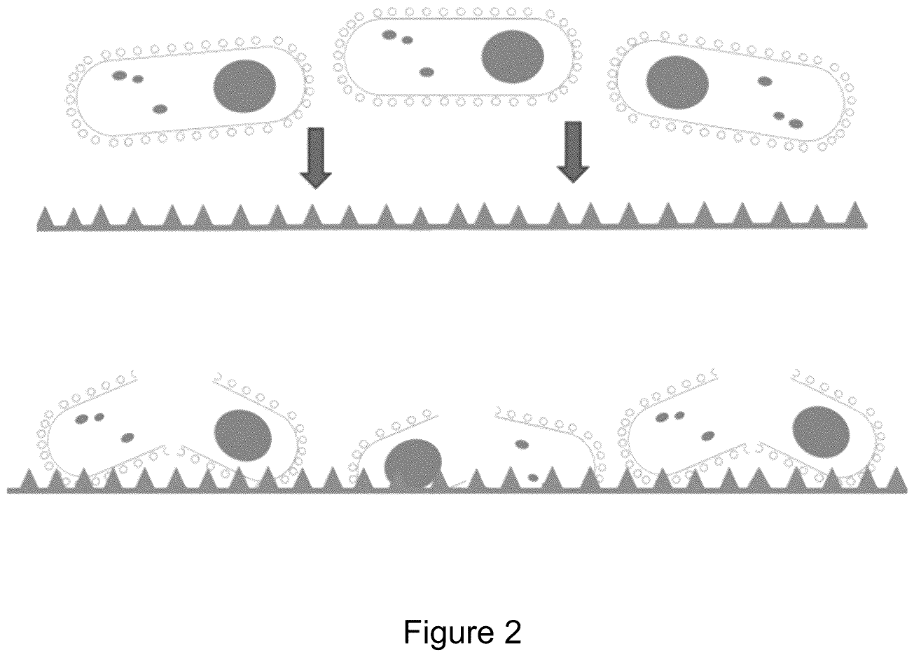

To encourage sustainability and increased aerosol particle protection, the present studies developed a cellulose nanofiber/polyvinyl alcohol (CNF/PVA) water-based dispersion applied onto commercial/DIY fabric masks (FIG. 1). The water-soluble dispersion, comprising CNF and polyvinyl alcohol (PVA), was developed as a spray agent capable of covering surface of fabric masks to enhance protection and sustainability against harmful aerosol particles. PVA was chosen as the water-soluble binding agent to effectively adhere CNF onto the mask surface. The project followed the biomimics of dragonfly wings having uneven nanopillar surfaces to trap and rip bacterial membranes, as the spray decreased a water droplet contact angle on fabric surface resulting in an increase of adhesion for incident bacteria and/or viruses (FIG. 2). The CNF/PVA spray was also low-cost and biocompatible and enabled multi-use through home laundering. CNF/PVA water-soluble dispersion capable of being sprayed on commercial or homemade masks are also likely eligible for FDA medical mask or NIOSH N95 respirator testing standards (Goodge et al., 2023, AATCC Journal of Research, 10:18-27).

Cellulose plays an important role in all types of different media because it is the most abundant, biodegradable, renewable, and recyclable biopolymer in existence (Hongxia et al., 2020, Journal of the Science of Food and Agriculture, 100:4390-4399). Looking at the demand for cellulose from a systemic composition, it has a tight crystal structure, which is enclosed with hemicelluloses and lignin (Hongxia et al., 2020, Journal of the Science of Food and Agriculture, 100:4390-4399). These endless chains contribute to the tensile strength and durability of the molecule, which further generates plant cell walls. As well as increasing surface area, nanocellulose combines the key properties of cellulose, such as high specific strength and modulus, hydrophilicity, and extensive ability for chemical modification (Kargarzadeh et al., 2018, Cellulose, 25:2151-2189).

The use of cellulose nanofibers brings numerous benefits including renewability, widespread availability, low density, excellent mechanical properties, economic value, biocompatibility, and biodegradability (Kargarzadeh et al., 2018, Cellulose, 25:2151-2189). The fibers are extracted using chemical processes to further apply them to experiments as nanofibers. Nanocellulose offers a range of benefits across various industries, including food films, wound care, paper, and cotton-based textiles. For example, they can be used as a natural and biodegradable alternative to plastic in food packaging or as a wound dressing to promote healing and reduce scarring (Kwak et al., 2021, Carbohydrate Polymers, 258:117688; Gajjar et al., 2013, Retrieval studies for medical biotextiles. Woodhead Publishing, 182-210; Lin et al., 2020, Cellulose, 27:2651-2667; Ullah et al., 2020, International Journal of Biological Macromolecules, 155:479-489). In the paper industry, they can improve the strength and stability of paper products, and in the textile industry, they can enhance the durability and performance of cotton-based fabrics (Mirmehdi et al., 2018, Journal of Wood Chemistry and Technology, 38:3233-3245; Kwiczak-Yigitbai et al., 2020, ACS Sustainable Chemistry & Engineering, 8.10.1021).

PVA is a polyhydroxy water-soluble polymer. PVA is also biodegradable and biocompatible in many biomedical applications, including surgical sponges, orthopedic stabilization splints, blood-contacting material, etc. (Zulkifli et al., 2013, Procedia Engineering, 53:689-695). PVA aids in forming low-cost, hydrophilic, and transparent films with exceptional adhesion properties (Rusu et al., 2007, Journal of Optoelectronics and Advanced Materials, 9:1044-1047).

CNF and PVA were able to work together in water to form a gel-like material that was easily able to be constructed and sprayed in a variety of applications, such as coatings, adhesives, and films. This highly water-soluble material was easy to dissolve and remove through a simple washing technique like at-home laundering making it a sustainable multi-use product able be reapplied after each laundering cycle. Creating a CNF/PVA water-soluble emulsion that can be sprayed on commercial or homemade masks makes them eligible for FDA medical mask or NIOSH N95 respirator testing standards (Goodge et al., 20203, AATCC Journal of Research, 10:18-27).

This study put forth the idea of a CNF/PVA water-based dispersion spray applied onto commercial/DIY fabric masks, as a refined approach to foster sustainability and heightened protection against aerosol particles. There were no previous investigations into the impacts of using a handheld CNF/PVA spray (Naik et al., 2022, ACS Omega, 7:43559-43573; Singh et al., 2018, International Journal of Biological Macromolecules, 107:1879-1887; Meng et al., 2023, Composites Science and Technology, 232:109885). The present studies mark the first time this CNF/PVA deposition technique, employing a handheld spray bottle, has been evaluated. This method was a departure from others, such as a manual coater or airless spray pump (Huang et al., 2022, Sci Rep, 12:16148; Nadeem et al., 2023, Waste Biomass Valor, 1-14). A handheld spray bottle was chosen for its portability and user-friendliness. CNF/PVA were selected specifically due to their unique properties and potential.

This present studies also compared the CNF/PVA spray to NIOSH N95 mask standards by examining the spray's antibacterial properties following the biomimic concept of dragonfly wings (FIG. 2). In previous studies, it was discovered that nanopillars on the surface of insect wings contribute to the bactericidal property by individual cell penetration (Kamarajan et al., 2020, AMB Expr, 10:85; Ivanova et al., 2012, Small, 20:2489-2494). Individual bacterial cells were observed to be killed within three minutes of contact with insect wings, which were made of chitin with chemical properties similar to cellulose (Kamarajan et al., 2020, AMB Expr, 10:85; Ivanova et al., 2012, Small, 20:2489-2494; Quirk et al., 2013, Nature).

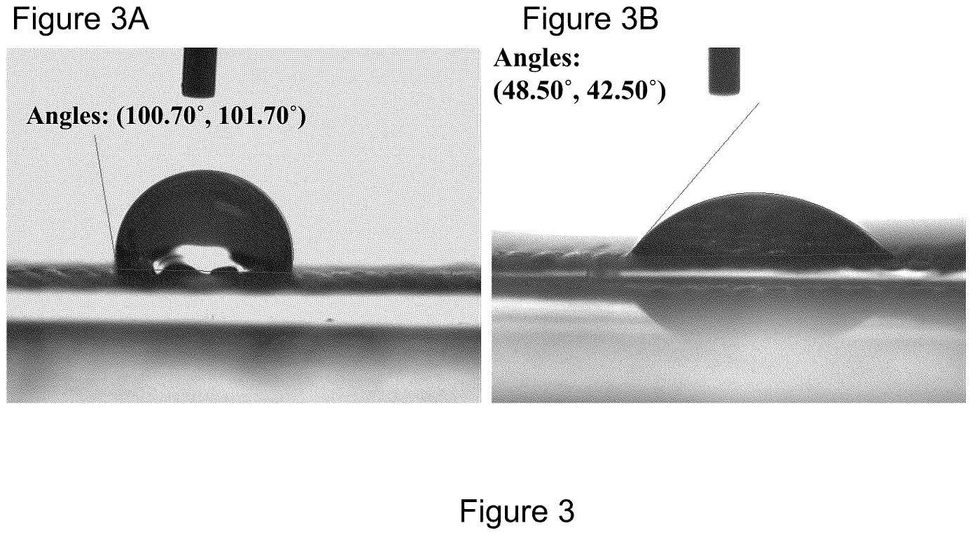

To evaluate the CNF/PVA spray, common synthetic and biodegradable fabrics were tested with instrumental measures, such as contact angle testing, optical microscopy, scanning electron microscopy (SEM), machine laundering, textile porosity testing, and bacterial filtration efficiency (BFE) testing (FIG. 3 through FIG. 10). To be comparable to NIOSH N95 mask standards, CNF/PVA sprayed masks must be evaluated according to the United States standards for filtration efficiency (ASTM F2100 and ASTM F2101-19). The development of a handheld CNF/PVA spray is novel concept and it opens up possibilities for additional enhancements as a new method for providing biocompatible and portable antibacterial protection.

Testing the Contact Angle with the VCA Optima XE

The contact angles were measured using the VCA Optima XE instrument. The average of six individual values recorded on each fabric was taken to congregate a mean and standard deviation value of both the right and the left angle measurements. Each fabric was sprayed five times with a drying time of one hour.

Cotton #1 demonstrated an increase in wettability by 16.150 with the CNF/PVA spray application and polyester demonstrated an increase in wettability by 42.9° largely greater than the cotton #1 textile. Nylon demonstrated the highest increase in wettability by a decrease of 48.70° in the contact angle out of the measured fabrics, indicating that there was an increased hydrophilicity to the surface with the spray applied (FIG. 3A and FIG. 3B). The polypropylene fabric demonstrated a decreased contact angle by 45.55° and the silk fabric demonstrated a decreased contact angle by 16.10°.

The increase in surface roughness and —OH presence of the CNF/PVA layering significantly impacted the morphological changes of the fabric surface and therefore enhanced the fabric samples' hydrophilicity, similar to the effect of a previous approach (Chen et al., 2021, ACS Applied Nano Materials, 10.1021). There was maximum adhesion on the nylon masks by the greatest decrease in contact angle, which indicated nylon contained the highest wettability properties. The contact angle decrease was attributed to an increase in ions at the surface and less compact structure with increased water absorption, which was comparative to previously published data (Kwak et al., 2021, Carbohydrate Polymers, 258:117688). Moreover, the results represented in Table 1 indicated that this development extended to most textiles regardless of their biodegradability.

TABLE 1
Contact Angle P-Values from ANOVA Analysis (Probability of obtaining
an F-statistic greater than the computed F); Filled Cells:

| Source of Variation | Contact Angle |
|---|---|
| Spraying | <0.01** |
| Fabric Comparison | <0.01** |

** significant. Spraying (degrees of freedom (df): 11, F: 14) Fabric Comparison (df: 4, F: 55).

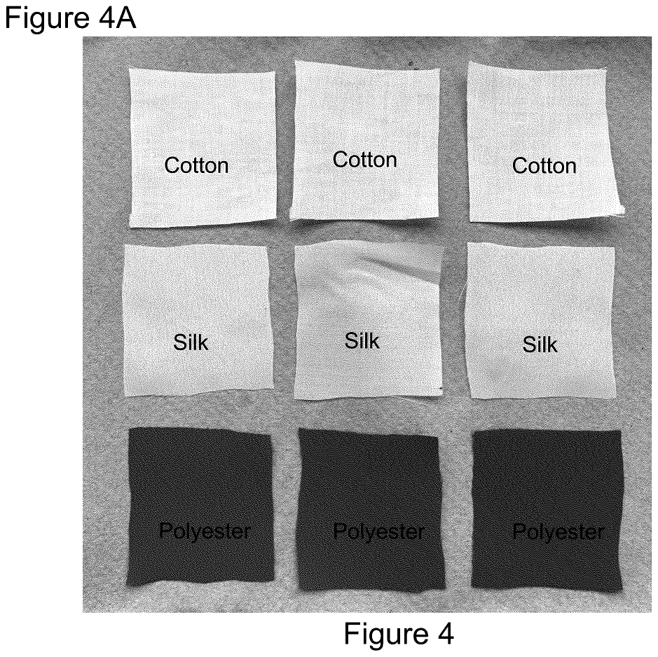

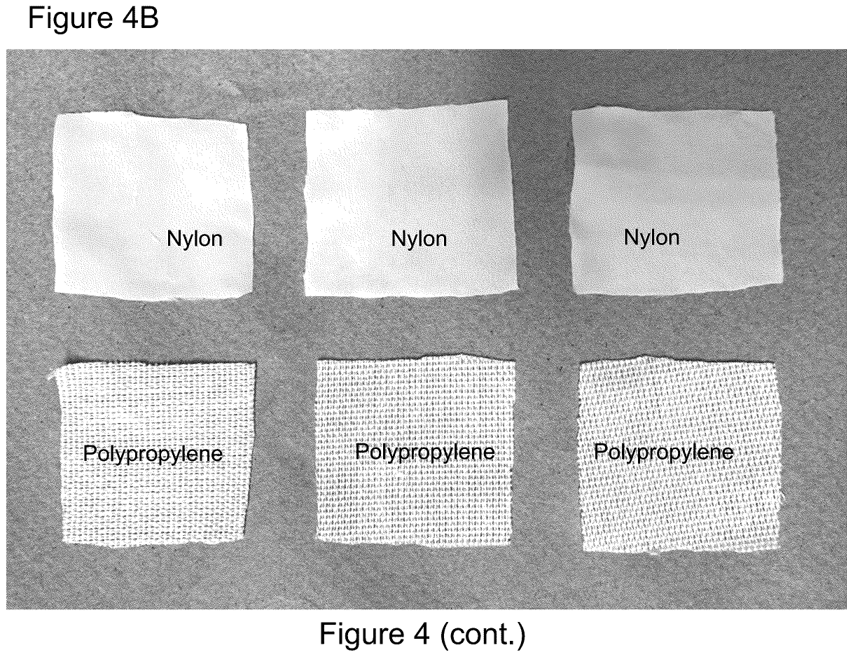

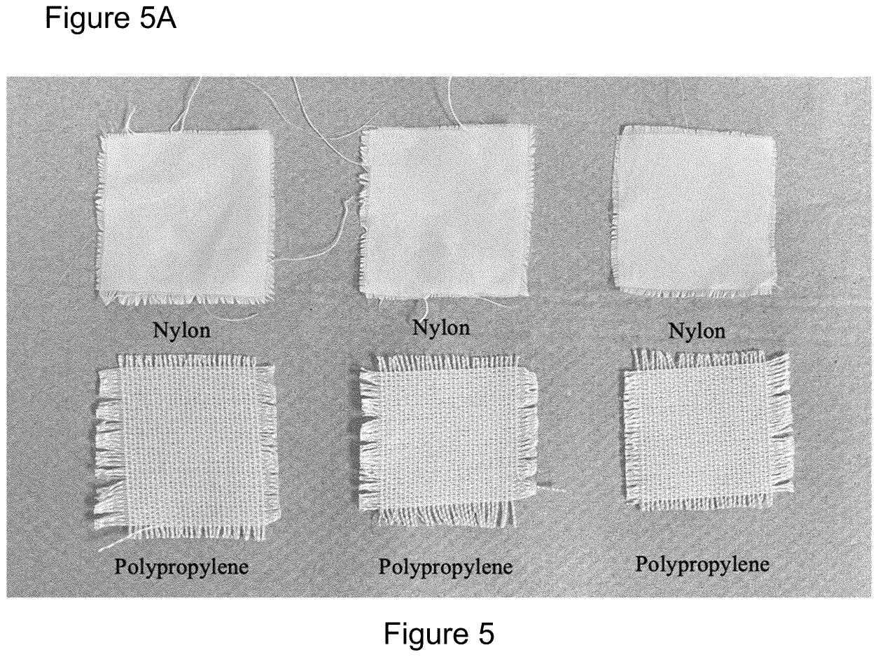

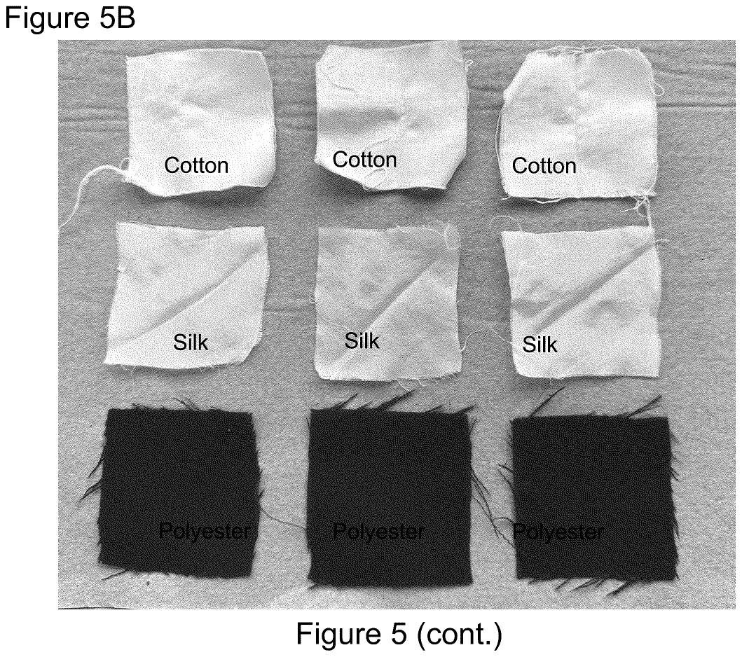

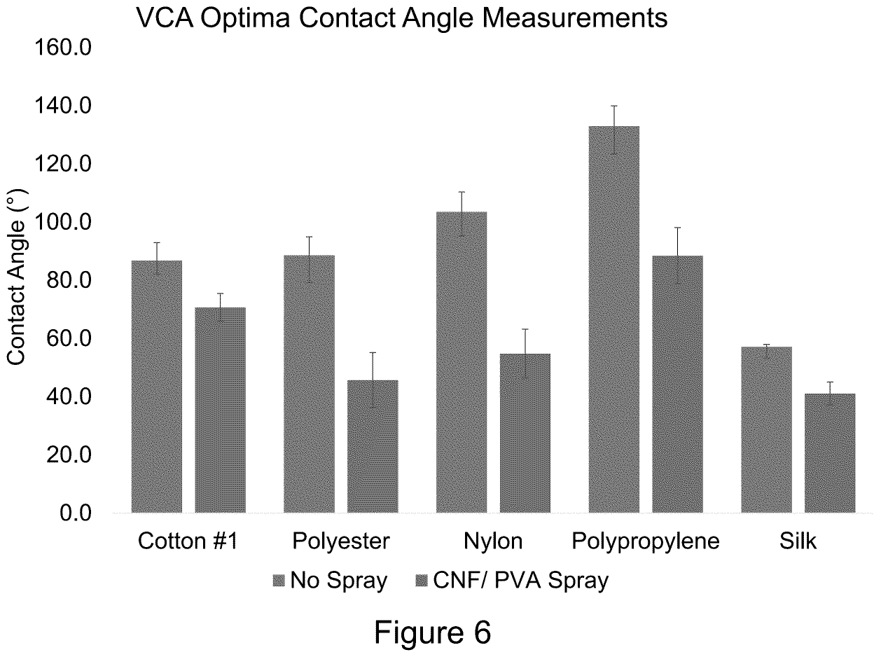

FIG. 6 demonstrated the significance of the CNF/PVA addition by displaying a two-way ANOVA statistical analysis measuring variance between the five different textiles (FIG. 4A, FIG. 4B, FIG. 5A, and FIG. 5B). The uncertainties in the standard error bars represent individual standard deviation (FIG. 6). Different textiles were utilized to measure the increased hydrophilic effects across various diameters of porous areas and various fiber makeups, which demonstrated CNF/PVA adaptability. Before statistical analyzation, the data was checked and evaluated for normal distribution.

Polyester, a synthetic fabric usually derived from petroleum, is normally characterized by its large surface area per unit mass and small pore size (sewport.com/fabrics-directory/polyester-fabric#:~:text=Polyester%20is%20a%20synthetic%20fabric,within%20the%20ester%20functional%20group; Azeem et al., 2018, Vlakna a Textil, 25). Nylon, a synthetic fabric produced from coal contains hydrophilicity resulting from the amide linkage can forming with hydrogen bonds and water molecules in the amorphous regions (Hench et al., 2005, Biomedical polymers. In: Biomaterials, Artificial Organs and Tissue Engineering, Woodhead Publishing Series in Biomaterials, 97-106). Cotton #1, one of the most abundantly used biodegradable fabrics, exhibits high moisture-wicking and breathability (Salma et al., 2020, Heliyon, 2020, 6:8). Polypropylene, a synthetic fiber derived from a thermoplastic polymer of the polyolefin group is used in many medical textiles (including standard surgical masks) with its microporous membrane and extreme hydrophobicity (sewport.com/fabrics-directory/polypropylene-fabric; Ariono et al., 2017, IOP Conference Series Materials Science and Engineering, 214:10). Silk is classified as high molecular weight organic polymers, exhibiting characteristic repetitive peptide sequences with hydrophilic properties (Murugesh Babu et al., 2013, Structural Aspects of Silk, Woodhead Publishing, 56-83).

Overall, the various fabrics exhibited a high statistical significance regarding the CNF/PVA addition two p-values of <0.01. Regarding spraying, one p-value of <0.01 (Table 1) demonstrated the effect of the CNF/PVA spray between the control and experimental samples, with the significance confirming the change of ion structure at the surface of the textiles. Regarding fabric comparisons, p-value of <0.01 (Table 1) demonstrated the statistical effect of each fabric to one another, with significance confirming the differences in chemical makeup of the fiber formation.

Examining Surface Morphologies with Nikon Ni-E Upright Motor

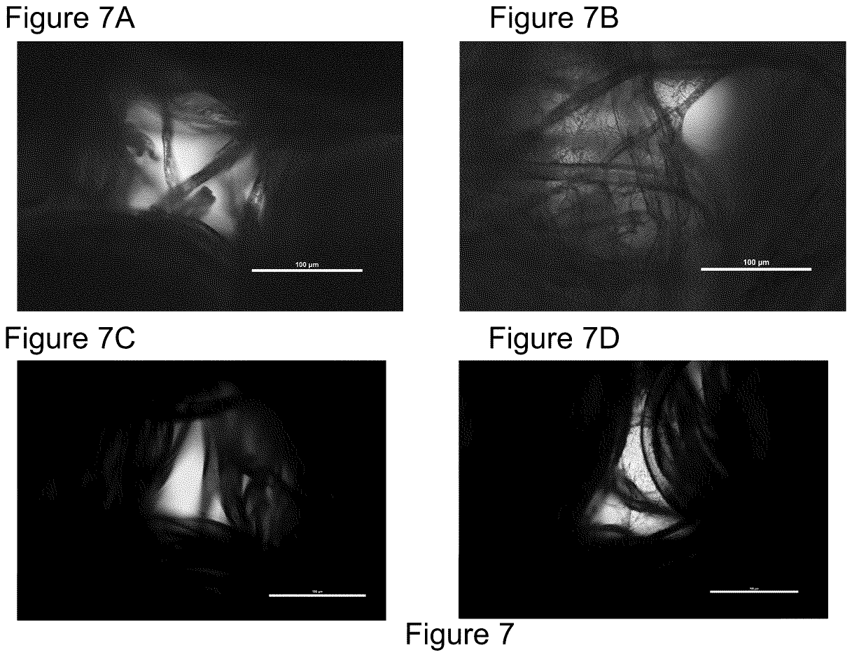

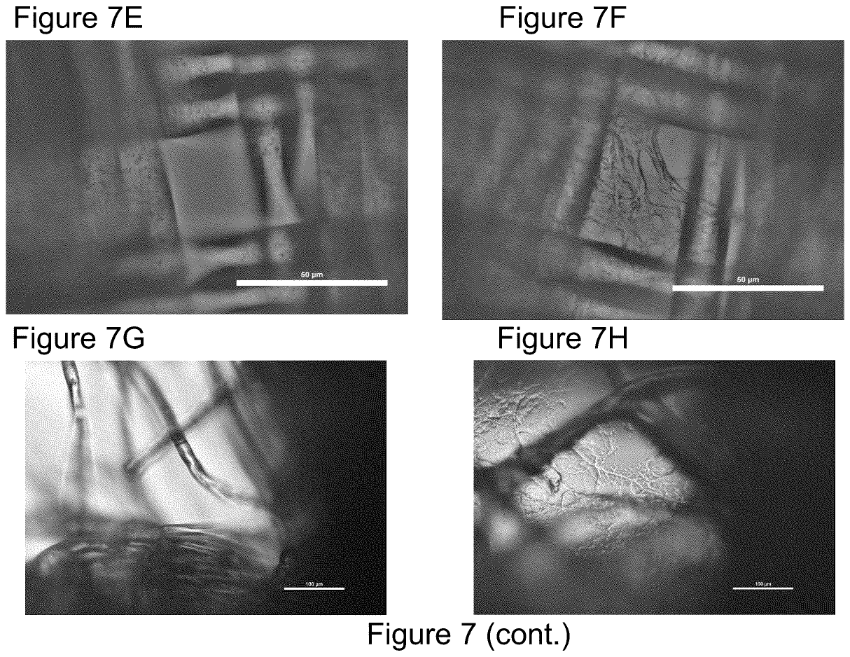

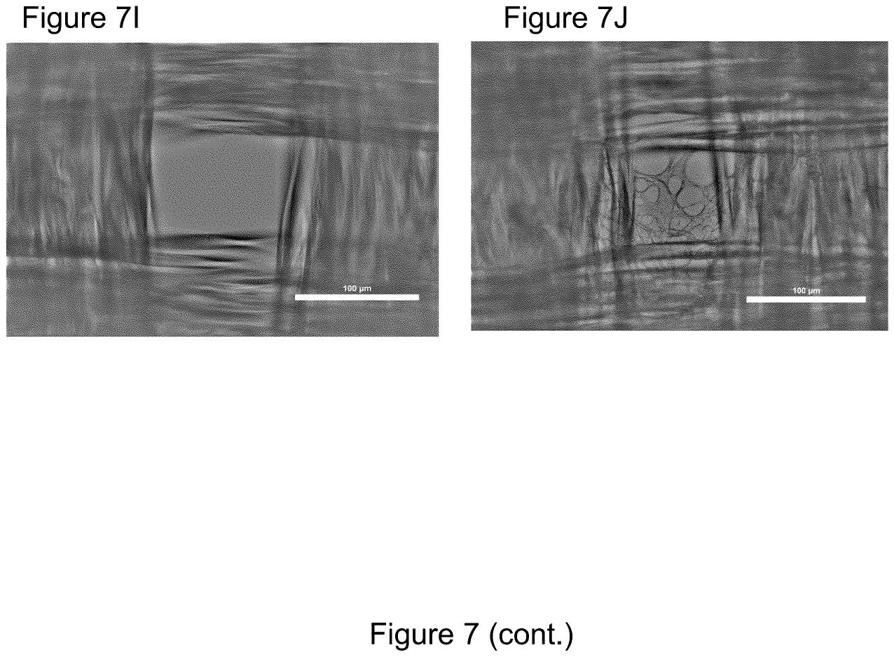

As shown in FIG. 7, the Nikon Ni-E Upright motor was used to capture images of the surface morphologies of uni-layered cotton #1, polyester, nylon, polypropylene, and silk fabrics, both without and coated with a CNF/PVA spray. The cotton #1, polyester, and polypropylene images were taken were taken with a magnification of 10× using HDR 3-layering and differential image contrast (FIG. 7A, FIG. 7B, FIG. 7C, FIG. 7D, FIG. 7G, and FIG. 7H). The nylon images were taken with a magnification of 40× using HDR 3-layering and differential image contrast (FIG. 7E and FIG. 7F).

The CNF/PVA coating was clearly visible in the porous areas of the fabric and generated a physical barrier that helped reduce the transmission of aerosols and changed the surface roughness of the exposed outer area of the fibers present. The silk images were taken with a magnification of 20× using HDR 3-layering and differential image contrast (FIG. 7I and FIG. 7J).

Regardless of the fabric type, the CNF/PVA coating was similarly present on the porous areas, reducing the size of the pores across the entire fabric. However, the experimental images showed a non-uniform layer of CNF/PVA. The uneven distribution of the CNF/PVA on the fabrics was likely due to factors such as the size, shape, and velocity of the droplets produced when the water-soluble dispersion is sprayed. This, however, did affect the overall amount of CNF/PVA applied to the textiles as there was a similar film deposition throughout, which was evident by the below described experiments.

Examining Surface Morphologies with SEM

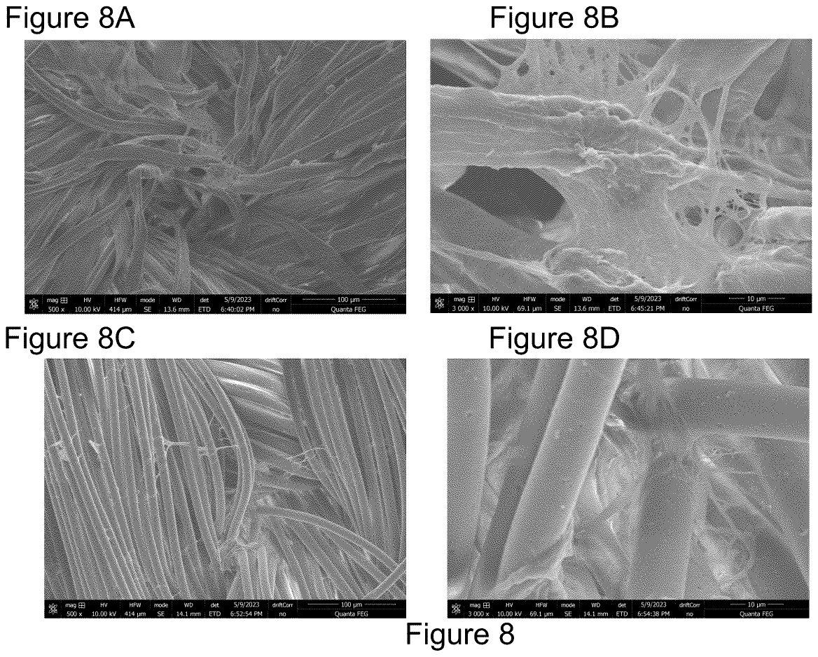

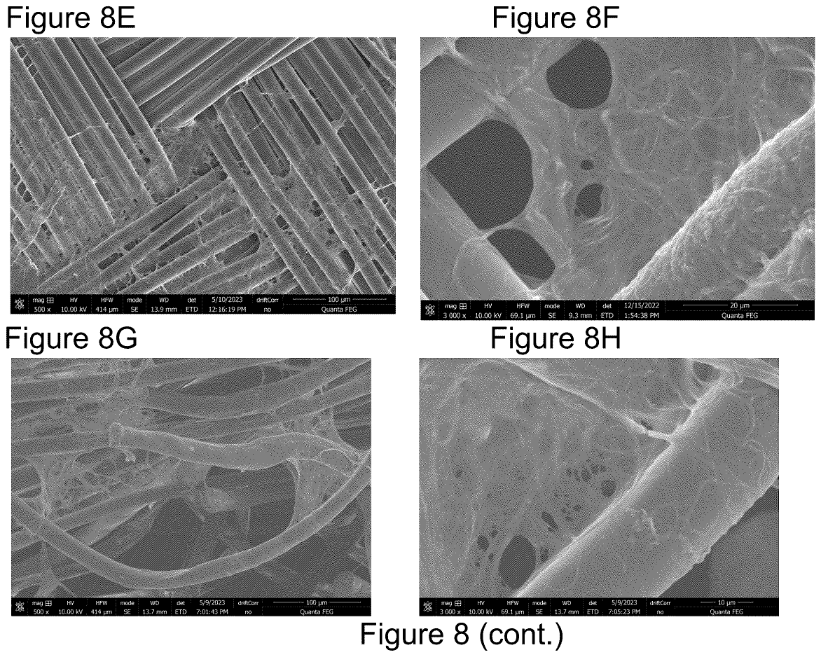

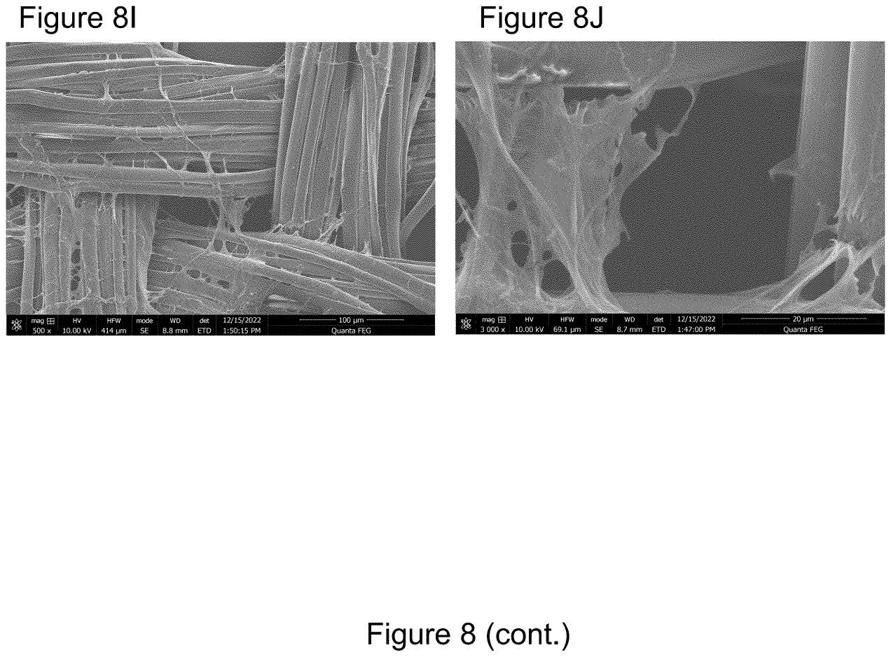

As shown in FIG. 8, various images FEI Quanta 650 SEM of cotton #1, polyester, nylon, polypropylene, and silk fabrics were collected after they were sprayed 5 times with CNF/PVA spray with optimal drying time. Within each captured photo, fiber surface area increased as it was layered with the CNF/PVA spray film. Individual cellulose nanofibers were clearly identified through the PVA film among each textile fiber and porous area regardless of whether they were synthetic or biodegradable fibers. All textiles were examined at two different magnifications of 500× and 3,000×, which present a textile cross section and fabric porous area (FIG. 8A through FIG. 8J).

CNFs were dispersed from fiber to fiber while also encompassing individual fibers. This generated an increased physical defense against aerosol particles. CNF/PVA spray film visually adhered within and on the surface of the textile fibers as a rugged-like texture. These surface morphologies were similar to ones previously reported and contained uneven, sharp edges on the substrate surface that supported the biomimic concept that physical cellulosic barriers increase antibacterial resistance (Tyagi et al., 2019, Langmuir: the ACS journal of surfaces and colloids, 35:104-112).

Machine Laundering Capabilities with the Launder-O-Meter

Table 2 lists the contact angle measurements obtained using the VCA Optima XE instrument, and the average of ten individual values was taken to congregate a mean value for both pre- and post-laundered samples on cotton #1, polyester, nylon, polypropylene, and silk.

TABLE 2
Surface Contact Angles for Cotton #1, Polyester, Nylon, Polypropylene,
and Silk fabric samples, both Pre- and Post-laundering.

| | No Spray Samples | | CNF/PVA Sprayed | |
|---|---|---|---|---|
| | Mean (degree) | | Samples Mean (degree) | |
| | Pre- | Post- | Pre- | Post- |
| Fabric | Laundering | Laundering | Laundering | Laundering |
| Cotton #1 | 86.9 | 57.0 | 87.3 | 59.1 |
| Polyester | 85.6 | 61.5 | 79.1 | 61.9 |
| Nylon | 118.6 | 94.8 | 113.4 | 96.5 |
| Polypropylene | 136.2 | 130.9 | 133.9 | 133.94 |
| Silk | 50.2 | 32.2 | 48.1 | 40.0 |

CNF/PVA experimental samples were sprayed five times after the contact angle measurements were recorded to test if CNF/PVA left residual residue post-machine laundering. Machine laundering was conducted with the Launder-O-Meter with simulated abrasion to replicate an at-home washing cycle. To further evaluate CNF/PVA spray adhesion, a two-way ANOVA statistical analysis was conducted for each textile, and results are present in Table 3. Before statistical analyzation, the data was checked and evaluated for normal distribution.

TABLE 3
Machine Laundering P-Values from ANOVA Analysis (Probability of obtaining an F statistic greater than
the computed F); Filled Cells: **significant. Contact Angle (CA); Weight (W); Spraying (S); Washing
(Wa); Interaction (I). Cotton 1: CA:S (df: 1.00, F: 0.27), Wa (df: 1.00, F: 145.98), I (df: 36, F:
0.13) W:S (df: 1.00, F: 7.99), Wa (df: 1.00, F: 0.56), I (df: 1.00, F: 2.23), Polyester: CA:S (df:
1.00, F: 0.89), Wa (df: 1.00, F: 41.28), I (df: 36, F: 1.16); W:S (df: 1.00, F: 3.18), Wa (df: 1.00,
F: 1.61), I (df: 1.00, F: 0.00); Nylon: CA:S (df: 1.00, F: 0.65), Wa (df: 1.00, F: 87.5), I (df: 1.00,
F: 119.70); W:S (df: 1.00, F: 4.12), Wa (df: 1.00, F: 26.13), I (df: 1.00, F: 3.15); Polypropylene:
CA:S (df: 1.00, F: 0.05), Wa (df: 1.00, F: 2.31), I (df: 1.00, F: 2.38); W:S (df: 1.00, F: 0.35), Wa
(df: 1.00, F: 88.3), I (df: 1.00, F: 0.49); Silk: CA:S (df: 1.00, F: 0.70);, Wa (df: 1.00, F: 23.52),
I (df: 1.00, F: 0.12); W:S (df: 1.00, F: 0.28), Wa (df: 1.00, F: 1.03), I (df: 1.00, F: 0.46)

| | Cotton 1 | | Polyester | | Nylon | | Polypropylene | | Silk | |
|---|---|---|---|---|---|---|---|---|---|---|
| Source of | Contact | | Contact | | Contact | | Contact | | Contact | |
| Variation | Angle | Weight | Angle | Weight | Angle | Weight | Angle | Weight | Angle | Weight |
| Spraying | 0.61 | 0.01 | 0.35 | 0.09 | 0.42 | 0.06 | 0.82 | 0.56 | 0.41 | 0.60 |
| Washing | <0.01** | 0.45 | <0.01** | 0.22 | <0.01** | <0.01** | 0.14 | <0.01** | <0.01** | 0.32 |
| Interaction | 0.72 | 0.16 | 0.29 | 0.95 | 0.12 | 0.12 | 0.13 | 0.49 | 0.12 | 0.51 |

Table 4 demonstrates weight as variable that affected the overall textile property based on CNF/PVA adhesion through machine laundering.

TABLE 4
Weight Measurements for Cotton #1, Polyester, Nylon, Polypropylene,
and Silk fabric samples, both Pre- and Post-laundering.

| | No Spray Samples | | CNF/PVA Sprayed | |
|---|---|---|---|---|
| | Mean (g/m²) | | Samples Mean (g/m²) | |
| | Pre- | Post- | Pre- | Post- |
| Fabric | Laundering | Laundering | Laundering | Laundering |
| Cotton #1 | 128.6 | 120.9 | 132.7 | 107.9 |
| Polyester | 161.2 | 151.9 | 168.4 | 158.4 |
| Nylon | 63.1 | 58.1 | 62.8 | 55.6 |
| Polypropylene | 160.2 | 134.7 | 156.9 | 135.2 |
| Silk | 34.2 | 34.1 | 34.4 | 35.1 |

Ten samples of cotton #1, polyester, nylon, polypropylene, and silk as 2×2 inch squares were measured on the precision scale. After measurements, five layers of CNF/PVA spray were applied to each five textile samples. Optimal drying time of at least one hour was allowed for the experimental samples in ambient air. Machine laundering was conducted with the Launder-O-Meter with simulated abrasion to replicate an at-home washing cycle, and after a 45-minute cycle samples were air-exposed for at least 24 hours for complete dehydration. To further evaluate CNF/PVA spray adhesion regarding weight, a two-way ANOVA statistical analysis was conducted (Table 3). Before statistical analyzation, the data was checked and evaluated for normal distribution.

Focusing on spraying through contact angle measurement, all textiles demonstrated p-values>0.05 (Table 3) regarding their pre-laundering contact angles compared to post-laundering contact angles. These results further indicated that if the textile fabric followed a controlled machine-washing cycle, the surface wettability and weight of fabric treated with CNF/PVA spray did not exhibit significant differences compared to untreated fabric post-laundering due to the hydrophilic properties of CNF and PVA. Similarly, post-laundering showed that the contact angles of the nylon and polypropylene experimental samples did not have a significant reduction, indicating that washing did not cause a dramatic surface tension change for those applied with the CNF/PVA spray. This indicated the complete removal of the CNF/PVA adhesion to textiles within one machine laundering cycle comparable to samples with no spray.

Focusing on washing through contact angle measurement, polypropylene demonstrated a p-value of >0.05 and cotton #1, polyester, nylon, and silk demonstrated p-values of <0.05 (Table 3). These results indicated that polypropylene contact angle remained similar before and after laundering and did not have a significant effect on hydrophobicity after one cycle of laundering. On the contrary, the contact angles of pre-laundering cotton #1, polyester, nylon, and silk were significantly higher than post-laundering contact angles, meaning machine laundering was a statistical factor affecting hydrophobicity.

Increasing mechanical abrasion of nylon during laundering resulted in significant differences in wettability of before and after a cycle, which caused fibers to shed from the fabric surface (Frost et al., 2020, AATCC Journal of Research, 7:32-41). This physical structure change can be referenced in FIG. 4 and FIG. 5.

To evaluate the impact of machine laundering of the two independent variables, the interaction between spraying versus washing were recorded in relation to the contact angle measurement. In a two-way ANOVA analysis, there was no notable interaction effect between spraying and washing on the contact angles for each fabric type. The variables of spraying and washing were independent, exhibiting no interactive effects, and showed consistent differences attributed only to the spraying or washing processes. These results indicated that the intended factor to change was the complete removal of the CNF/PVA spray through a single washing cycle.

Focusing on spraying through weight measurement, all textiles demonstrated p-values>0.05 (Table 3) regarding their pre-laundering weight compared to post-laundering weight. Post-laundering showed that the measured weight of the cotton #1, polyester, nylon, polypropylene, and silk experimental samples did not have a significant change, indicating that washing did not cause a dramatic weighted change for those applied with the CNF/PVA spray compared to those applied with no spray. These results indicated the complete removal of the CNF/PVA adhesion to textiles within one machine laundering cycle.

In another section of the two-way ANOVA analysis, the weighted effect was tested between the pre- and post-laundering samples aiming to measure if weight significantly changed before and after one cycle including spray presence. Focusing on washing through weight measurement, cotton #1, polyester, and silk demonstrated p-values of >0.05, which showed there was no significant difference in weight for both no spray and CNF/PVA samples. Nylon and polypropylene demonstrated p-values of <0.05 (Table 3), which confirmed a significant difference in weight for both no spray and CNF/PVA samples. These results are consistent with previous studies, which reported that that the washing process used in their study resulted in a decrease in the average molecular weight, and subsequently, a reduction in the elongation at breakage (Tapia-Picazo et al., 2014, Fibers and Polymers, 15:547-552).

To evaluate the impact of washing of the two independent variables, the interaction between spraying versus washing were recorded in relation to the weight measurement. In a two-way ANOVA analysis, there was no notable interaction effect between spraying and washing on the weight for each fabric type. The variables of spraying and washing were independent, exhibiting no interactive effects, and showed consistent differences attributed only to the spraying or washing processes. These results indicated that the intended factor to change was the complete removal of the CNF/PVA spray through a single washing cycle.

Subsequent studies focused on analyzing each textile for the spraying and washing effect. Contact angle and weight each played a role in determining the longevity of the textile in two separate ways. Spraying evaluated the CNF/PVA film adhesion through machine laundering and increased abrasion. The hydrophilic nature of the CNF/PVA film contributed to the complete removal through one laundering cycle, which made it more attractive to restart the cycle of daily mask usage and continue the reapplication of CNF/PVA film.

Textile Porosity Testing with the Advanced Capillary Porometer (PMI)

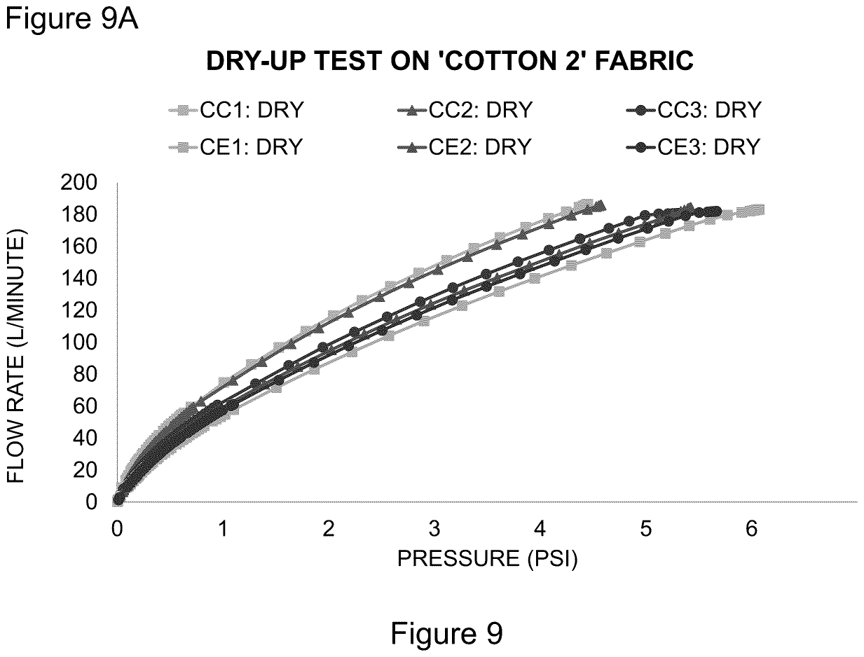

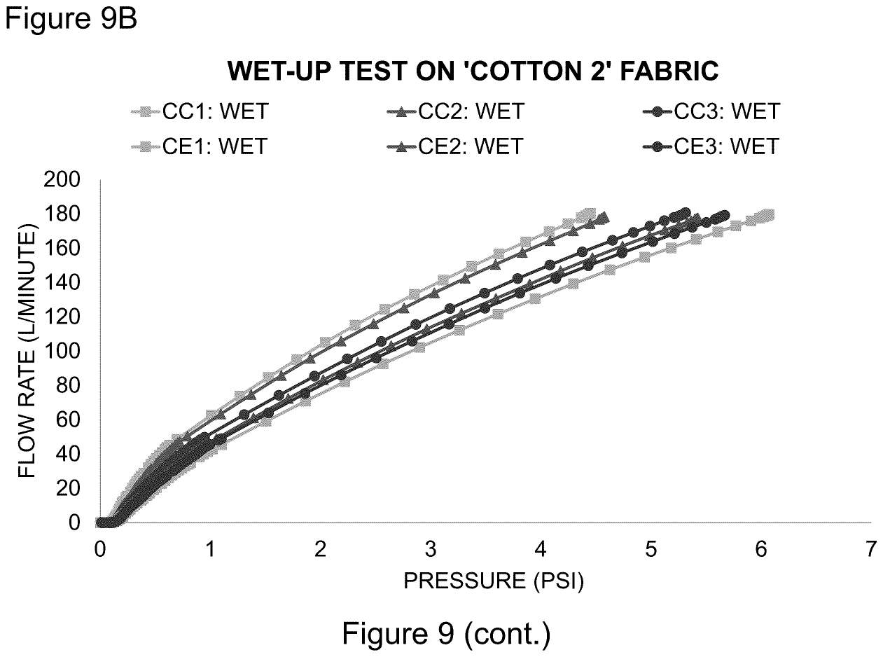

The Dry-Up/Wet-Up extraction method conducted with the Advanced Capillary Porometer was utilized to compare porous differences between cotton 2 fabric and cotton 2 CNF/PVA layered fabric shown in FIG. 9. Cotton 2 fabric was used to simulate CNF/PVA spray application to a commercialized mask, which contained 3-layered cotton fabric. Particle filtration measured with Dry-Up/Wet-Up testing examined 3 control and 3 CNF/PVA textiles to limit testing discrepancies.

FIG. 9 represents both parameters of the Dry-Up/Wet-Up testing method with increasing pressure values correlating to increased flow rate (L/minute). The initial intersection of all the linear models corresponds to the maximum pore size, also known as bubble point. The ending points of all linear models on the Dry-Up (FIG. 9A) and Wet-Up (FIG. 9B) graphs correspond to the minimum pore size that includes all the liquid resulting in the porous areas being emptied (Zhang et al., 2022, Journal of Industrial Textiles, 51:1372S-1391S). In both dry and wet parameters, there were visual discrepancies between the experimental and control fabrics, indicating that there was a measured difference in porous areas with application of the CNF/PVA spray.

As shown in Table 5, the control cotton demonstrated a mean flow pore diameter of the three measurements at 31.37 microns and a mean bubble point pore diameter of 650.16 microns.

TABLE 6
Control and Experimental (CNF/PVA spray)
Cotton Fabric Porosity on Cotton Fabric.

| | Mean | Bubble | Bubble | | Mean Flow |
|---|---|---|---|---|---|
| | Pore | Point Pore | Point | Maximum | Pore |
| Sample | Size | Diameter | Pressure | Pore Size | Diameter |
| ID* | (μm) | (μm) | (PSI) | Distribution | (μm) |
| CC1 | 27.82 | 137.97 | 0.06 | 71.99 | 30.76 |
| CC2 | 31.13 | 314.78 | 0.026 | 97.82 | 28.34 |
| CC3 | 59.84 | 1497.72 | 0.006 | 25.26 | 35.02 |
| CE1 | 31.36 | 167.94 | 0.05 | 14.07 | 24.21 |
| CE2 | 54.902 | 853.75 | 0.01 | 52.03 | 22.69 |
| CE3 | 136.63 | 3447.66 | 0.002 | 55.74 | 21.32 |

*CC1, CC2, CC3: Cotton Control Fabrics without CNF/PVA spray; CE1, CE2, CE3: Cotton Experimental Fabrics with CNF/PVA spray.

The experimental CNF/PVA cotton demonstrated a mean flow pore diameter of the three measurements at 22.74 microns and a mean bubble point pore diameter at 1489.78 microns. A correlation between the CNF/PVA application and decrease in porous diameter values was observed. An increase in bubble point pore diameter values was also observed. Bubble point pore diameter demonstrated a great fluctuation in values due to change in textile surface patterns. These results contrasted with the mean flow pore diameter in the inverse direction as comparable to the publication (Zhang et al., 2022, Journal of Industrial Textiles, 51:1372S-1391S). Additionally, maximum pore size distribution was recorded for the cotton 2 fabric samples.

To find the numerical value, pore distribution must be measured for its change of filter flow between wet and dry samples, and then calculated from the equations below.

$$Filter Flow Percentage = 100 \times \frac{Wet flow}{Dry flow}$$

Increasing Filter Flow=(Filter flow % (current)−Filter flow % (previous))

$$Poor Distribution = \frac{Increasing Filter flow \%}{Diameter ( previous ) - Diameter ( current )}$$

The mean of the cotton control values of maximum size pore distribution was 64.82 and the mean cotton experimental values of maximum size pore distribution was 40.61. These observations were representative of a differential change of filter flow between the control and experimental samples with the porous distribution being more miniscule. Maintaining a continuous flow through the Advanced Capillary Porometer further emphasized the importance of physical closure of fabric porous areas without diminishing total air permeability but decreasing aerosol leakage. Air permeability was important as masks are used as a breathing apparatus to keep the wearer maintain normal respirating levels, while continuously being protected from outer infectious inhalants.

In assessing air permeability and breathability, it was pivitol to draw insights from comparative studies. Akduman created cellulose acetate nanofiber mats for N-95 respirators and reported a mean flow pore size ranging from 2.38 μm-5.71 μm (Akduman et al., 2021, Journal of Industrial Textiles, 50:1239-1261). As such, adequate breathability can be accomplished even with smaller pore sizes with a range of 2.38 μm-5.71 μm.

### Evaluating Bacterial Filtration Efficiency

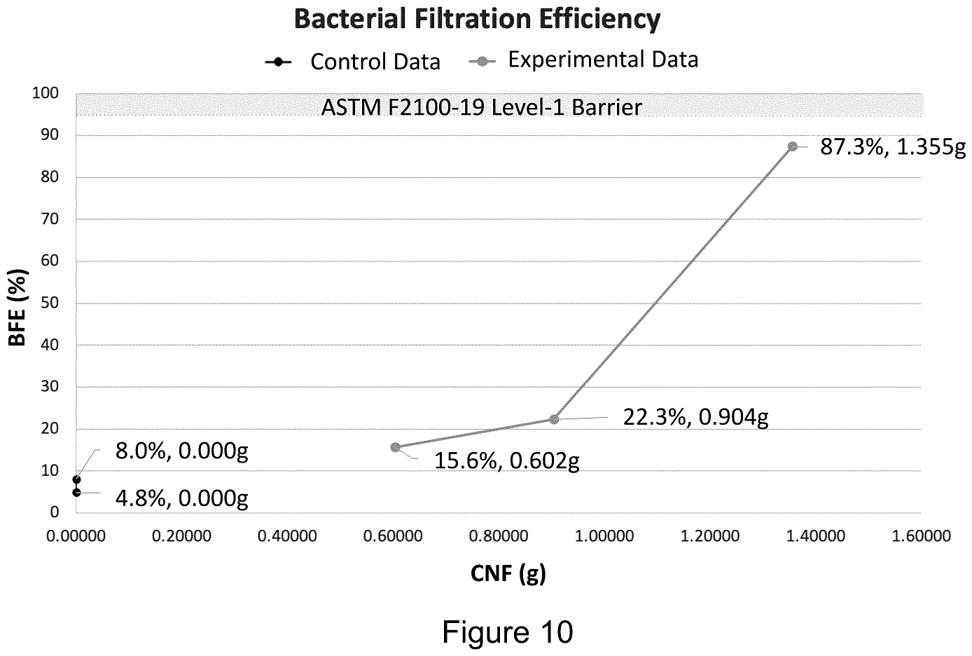

As shown in FIG. 10, the BFE of 5 cloth masks sprayed 5 times each were measured using the ratio of Staphylococcus aureus bacterial challenge from upstream to downstream. Cotton 2 fabric was used to simulate CNF/PVA spray application to a commercialized mask, which contained 3-layered cotton fabric in the form of a cloth face covering.

Concentrations through the samples consisted of three levels (0.602 g, 0.903 g, 1.355 g) of 3 wt % CNF and identical concentration of PVA (5 g of PVA along with additional 55 mL H₂O). To objectively evaluate the relationship between increasing CNF concentrations and associated BFE values, the Spearman rank correlation analysis was employed. The resulting coefficient of approximately “ρ”=0.97 demonstrated a strong positive value between the two variables.

### Spearman's Rank Correlation Coefficient

$$ρ = 1 - \frac{6 \sum {d}_{i}^{2}}{n ( {n}^{2} - 1 )}$$

Paired with a p-value of <0.01, it became evident that there was a statistically significant monotonic increase in BFE values with rising CNF concentrations. The sample size contained five Cotton 2 masks tested. Additional studies are performed with a larger sample size is recommended. There was an increased difference of bacterial properties in correlation with increasing CNF concentrations. These results indicated that CNF demonstrated antibacterial properties towards aerosol particles following the biomimic concept.

In the study, it was also found that the maximum CNF concentration (1.355 g of 3 wt % CNF) achieved a bacterial filtration efficiency value of 87.3% based on the BFE test result following ASTM F2100-19, which is the US standard specification for performance of materials used in medical facial masks (nelsonlabs.com/testing/bacterial-viral-filtration-efficiency-bfe-vfe/). This value remained close to the Level-1 barrier filtration (>95%).

Furthermore, as the CNF/PVA spray film was proven to work as an antibacterial agent with the escalating rise in BFE levels, this encompasses viruses as well. Viruses contain sizes ranging from 60 to 140 nm, and they can be expelled into ambient air similarly to bacteria through the inhalation of microdroplets larger than 5 μm and aerosols below 5 μm (Pardo-Figuerez et al., 2021, Nanomaterials (Basel), 11:900; Chan et al., 2013, Trends Microbiol, 10:544-555; Leung et al., 2020, September Purif. Technol, 250:116886). Thus, the CNF/PVA spray film is served dual purpose as an anti-bacterial agent and a robust physical barrier against aerosol particles.

In summary, various testing of biodegradable and synthetic fabrics were conducted to test for a positive correlation of decreased aerosol transmission with various instruments: VCA optima, Optical microscope, SEM, Machine Laundering, PMI, and BFE. Through testing the CNF/PVA water-soluble dispersion applied onto the mask fabrics, it was found that there was a significant correlation between the CNF/PVA loading and the hydrophilicity increase. The CNF/PVA spray changed surface texture, decreased porous areas, and improved the barrier property against aerosol bacteria.

The lower contact angles indicated there was significant stronger adhesion in the CNF samples due to the hydrophilic nature of cellulose and PVA. Post-laundering showed that the contact angles did not have a significant reduction in comparison with the pre-laundering contact angles, indicating that washing did not cause a dramatic surface tension change for fabrics applied with the CNF/PVA coating spray, which was the target outcome. Regarding the fabric weight reduction through washing, machine laundering was not affected by the addition of CNF/PVA spray on each of the textile samples, but differences in weight were from mechanical abrasion.

Regarding the optical microscope and SEM images, the CNF/PVA solution visually demonstrated increased adhesion. The PMI porosity testing revealed smaller mean flow pore diameters when the test fabrics were applied with the CNF/PVA spray to create an increased physical barrier against foreign particles. The test of BFE in accordance with ASTM F2100-19 revealed that the application of our CNF/PVA spray could increase BFE of the mask fabrics to 87.3% close to 95% for the professional mask with Level 1 barrier performance.

Overall, the study's positive results effectively showed the various capabilities of the CNF/PVA spray and substantiating the biomimicry concept. A significant strength of the study included antibacterial clearance against bacterial pathogens with an increase in mass percent, which was parallel of the previous findings, in which both studies use S. aureus at a test organism (Naik et al., 2022, ACS Omega, 7:43559-43573). Moreover, the present study tested five distinct textiles, both synthetic and biodegradable to evaluate the versatility of the spray's deposition on different textiles.

Additional testing of CNF/PVA antimicrobial spray applications are expanded to other forms of PPE, such as surgical gowns, caps, and air filters to limit aerosol particles. Additional research also expands on the spray's effectiveness against other bacterial species, such as E. coli, potential mold, foreign bodies, as well as viruses to further understand how the CNF/PVA spray can combat these microorganisms.

In conclusion, the utilization of face-covering masks as an extended form of PPE has led to exponential waste measures during the Covid-19 pandemic, with estimations of up to 7,200 tons of medical-type waste daily. A primary cause of this waste is surgical layered disposable masks that are constructed by melt-blown nonwovens usually made of non-biodegradable thermoplastic polymers such as polypropylene. To increase widespread sustainable options to the public, commercialized or DIY-based fabric masks serve as a solution, but their resistance to harmful molecules is less than the medical-grade masks due to their structure of fabrics, leaving space for penetration. This project examined a water-soluble dispersion composed of CNF and polyvinyl alcohol (PVA), as a spray agent capable of treating mask fabric surface to promote protection and sustainability against harmful aerosol particles. CNF spray is also low-cost and biocompatible and could allow multi-use through home laundering. PVA was chosen as the water-soluble bonding system to effectively adhere CNF onto the mask surface. This project followed the biomimics of dragonfly wings having uneven nanopillar surfaces to trap and rip bacterial membranes, as the spray decreases water droplet contact angle on fabric surface resulting in an increase of adhesion for incident bacteria and/or viruses.

The materials and methods employed in the present experimental example are now described.

### Materials

The compositions used a cellulose in the nanofiber slurry form of 3.0 wt % aqueous gel with a lateral dimension of 50 nm wide and lengths of up to several microns and PVA with a molecular weight of 85,000-124,000 Da and 95.596.5 hydrolyzed. PVA is at a concentration of 9.0 wt %.

A cellulose identification stain of Shirlastain D from SDL Atlas was applied to highlight the CNF on the fabric surfaces for microscopic examination.

CS in the fine powder form with specifications of 8500 deactetylation, which was the middle deacetylation degree ofLCS, was used.

Biodegradable fabric samples of 100% cotton (Cotton #1) and 100% silk used for testing were obtained from Testfabrics, Inc (Table 6).

TABLE 6
Fabric Descriptions.

| | | | Fabric | | |
|---|---|---|---|---|---|
| | | | Thick- | Fabric | |
| | Fiber | Fabric | ness | Weight | Instrument |
| Fabric ID | Content | Layers | (mm) * | (g/m²) ** | Utilized |
| Cotton #1 | 100% | 1 | 0.33 | 130.6 | VCA Optima, |
| | | | | | Optimal |
| | | | | | Microscope, |
| | | | | | Launder-O-Meter, |
| | | | | | SEM |
| Polyester | 100% | 1 | 1.33 | 164.8 | VCA Optima, |
| | | | | | Optimal |
| | | | | | Microscope, |
| | | | | | Launder-O-Meter, |
| | | | | | SEM |
| Nylon | 100% | 1 | 0.18 | 63.1 | VCA Optima, |
| | | | | | Optimal |
| | | | | | Microscope, |
| | | | | | Launder-O-Meter, |
| | | | | | SEM |
| Polypro- | 100% | 1 | 0.63 | 160.2 | VCA Optima, |
| pylene | | | | | Optimal |
| | | | | | Microscope, |
| | | | | | Launder-O-Meter, |
| | | | | | SEM |
| Silk | 100% | 1 | 0.14 | 34.7 | VCA Optima, |
| | | | | | Optimal |
| | | | | | Microscope, |
| | | | | | Launder-O-Meter, |
| | | | | | SEM |
| Cotton 2 | 100% | 3 | 1.10 | 406.0 | PMI, BFE |

* ASTM D1777 (ASTM D1777-96. ASTM International, West Conshohocken, PA, USA, 2019).
** ASTM D3776 (ASTM D3776/D3776M-09a. ASTM International, West Conshohocken, PA, USA, 2017).

Synthetic textile samples of 100% polypropylene, 100% polyester, and 100% nylon were used for testing (Table 6). To research a direct comparison to commercial masks, 100% cotton (Cotton 2) 3-layered masks were used. According to the packing information these masks might contain silver and/or copper. The inclusion of silver and/or copper might result in some antimicrobial properties. The packing information contained no claim regarding the mask's protectiveness against aerosol particles. The masks were not FDA approved; however, they have been authorized by the FDA under an Emergency Use Authorization (EUA) for use by Health Care Professionals (HCP) as (PPE) to help prevent the spread of infection of illness. The masks were machine washable and constructed of 100% cotton. The container holding the CNF/PVA dispersion was a glass reusable spray bottle (60 mL).

### Preparation of DI Water Soluble Dispersion of CNF and PVA

The product was designed to be a handheld bottle with a built-in spray function to coat various fabric surfaces. To generate a spray bottle with the most optimizable concentrations of CNF and PVA, a ratio of 0.066% CNF, 0.7% PVA, and 99% deionized water was used. This pertained to the pure and dry concentrations of CNF and PVA.

First, the 3 wt % CNF were removed from the refrigerator at 1-4 °C and 1.35531 g was measured on the scale. CNF were placed into a 55-mL beaker of DI water and spread evenly with an IKA RW 20 stirrer at a constant speed of 1000-2400 rpm.

Next, to prepare PVA, 5 g were measured on the scale and applied to a beaker with 50 mL of DI water. This generated a weighted concentration of 9 wt % PVA, which was later applied to the water-soluble dispersion after heating. The solution was heated using a hot bath of 175 °C for 45-60 mins until the bubbles were completely diminished.

5 mL of the PVA solution was measured and transferred to a beaker containing 55 mL DI water and CNF. Once distributed into a 60-mL spray bottle, the solution was shaken well and ready to be sprayed in its final homogenized state. The test fabric samples were sprayed from one foot away with five layers and an appropriate minimum dry time of 1 hour in ambient conditions. Before proceeding with testing, it was determined that applying a total of five layers of coating is the optimal approach to ensure uniform distribution of water-soluble spray across each textile fabric.

### VCA Optima XE

The VCA Optima XE was utilized to measure contact angles of water droplets against various test fabric surfaces (FIG. 3A and FIG. 3B). The contact angle of the liquid to the fabric measured the wettability of a solid surface by a liquid. An automated syringe of 100 μL was used and each measurement was taken by depositing a 2 μL droplet onto the textile surface. These contact angles were calculated automatically with the VCA Optima 2500 software system with a magnification of 35:1 and an accuracy of ±0.5°; each outcome was modified based on the surface tension of the fabric presented. The cotton #1, polyester, nylon, polypropylene, and silk fabrics were measured with control samples and CNF/PVA spray coated samples. Experimental textiles were sprayed with five layers and allowed for a drying time of at least one hour before testing proceeded.

### Optical Microscope

The Nikon Ni-E Upright Motor optical microscope was utilized in the Center for Biomedical Research Microscopy and Imaging Facility at UT to examine the fabric outer surface with CNF/PVA spray-film. The single layer pure cotton #1, polyester, nylon, polypropylene, and silk fabrics were viewed under the microscope without the CNF/PVA spray and with the CNF/PVA spray. All were chosen to identify if the CNF/PVA layering structure was similar in distribution on each fabric regardless of their woven structure or fiber chemical makeup.

### Scanning Electron Microscopy

The FEI Quanta 650 Scanning Electron Microscope (SEM) was utilized to evaluate the surface morphologies of cotton #1, polyester, nylon, polypropylene, and silk experimental fabrics. The machine contained voltage of 10.00 kV at a high vacuum and a chamber pressure of 0.60 mbar. The specimens were coated with gold before analyzation through the EMS Sputter Coater also located in the Texas Materials Institute.

### Launder-O-Meter

The Launder-O-Meter was utilized to simulate a controlled laundering environment for the CNF/PVA spray washability. Single layered cotton #1, polyester, nylon, polypropylene, and silk fabrics were laundered for a 45-minute cycle with 0.74 g of Seventh Generation natural laundry detergent for 24 total samples; each fabric sample contained three control samples and three experimental samples with 5 layers of CNF/PVA spray (FIG. 4A and FIG. 4B). Experimental samples were sprayed after measurement and left to dry in ambient conditions before the laundering cycle. Contact angles of the fabrics were measured with the VCA Optima XE to compare surface wettability before and after the Launder-O-Meter laundering. Fabric weight difference was another factor considered to test if textile weight significantly changes through machine laundering with the CNF/PVA spray applied. Experimental samples were sprayed after measurement and left to dry in ambient conditions before the laundering cycle.

### Advanced Capillary Porometer (PMI)

The Advanced Capillary Porometer (ACFP-1100AEXLFNBH) was utilized to characterize pore size and distribution with three control and three experimental measurements (CNF/PVA sprayed) on the cotton 2 fabric. Cotton 2 fabric was selected for its three-layered thickness, aligning with the instrument's requirement for a more substantial sample. Additionally, consistent with the other textiles examined in this study, these masks contained a 100% pure fiber composition. The highly reproducible technique required inputs of fabric diameter and fabric thickness. Out of five measurements of the fabric, an average thickness was calculated to be 0.0169 mm.

The Advanced Capillary Porometer followed the Dry-Up Wet Up testing procedure, which included monitoring the samples both in dry (before placement of liquid) and wet methods (after liquid placement). The test increased the pressure and measures the flow and pressure drop across the dry sample to locate the minimum and maximum detectable pore size on the cotton 2 fabric. The measured pressure curve was inversely proportional to pore diameter, which can be used to calculate pore size distribution (covalentmetrology.com/techniques/capillary-flow-porometry/). The surface tension of the Silwick fluid used for the samples is 20.1 Dynes/cm, which minimized likely evaporation during testing. Silwick fluid was chosen as it was the most optimal immiscible and saturated wetting liquid for this research in this field with its contact angle of zero (eurofins.com/consumer-product-testing/covid-19-product-testing/masks-respiratory-protective-devices/medical-surgical-masks/filtration-efficiency-testing-bfe-pfe/).

### Bacterial Filtration Efficiency Testing

The BFE testing was performed to determine the protective effectiveness of the commercial cotton masks (cotton 2) with the CNF/PVA spray against exterior bacteria. This testing measured the efficiency of the CNF/PVA sprayed fabric itself comparable to someone wearing a mask (pmiapp.com/wp-content/uploads/2019/12/1455264164-llp-1500a.pdf). When these results were reported, they were based off percentage, so the higher the percentage, the higher the efficiency. These results were aligned with the applicable standard values for the United States (ASTM F2100 and ASTM F2101-19), which made them essential to determine if this was a reliable commercial method.

While conducting the experimental procedures, the ratio of upstream bacterial challenge to downstream residual concentration determined the filtration efficiency of medical face mask materials. This method was specifically designed using Staphylococcus aureus as a challenge organism. Materials to be tested were conditioned at 21±5 °C and 85±5% Relative Humidity (RH) using a humidity chamber for 4 hours. The number of specimens were 5 in total with two control samples and three experimental (CNF/PVA spray) samples.

Cotton 2 fabric was selected for its resemblance to standard surgical masks commonly available to consumers. This choice endured a more direct comparison with regulated benchmarks. Furthermore, like the other textiles examined in this study, these masks contained a 100% pure fiber composition. Each experimental sample was sprayed 5 times and allowed for an ample amount of dry time in ambient air. The area of specimens tested was ~40 cm² and the temperature during testing was 26 °C. The mean particle size 3.0±0.3 μm and the flow rate was 28.3 L/min utilizing the GB-XF100 instrument.

### Example 2: PVA as an Adhering Agent

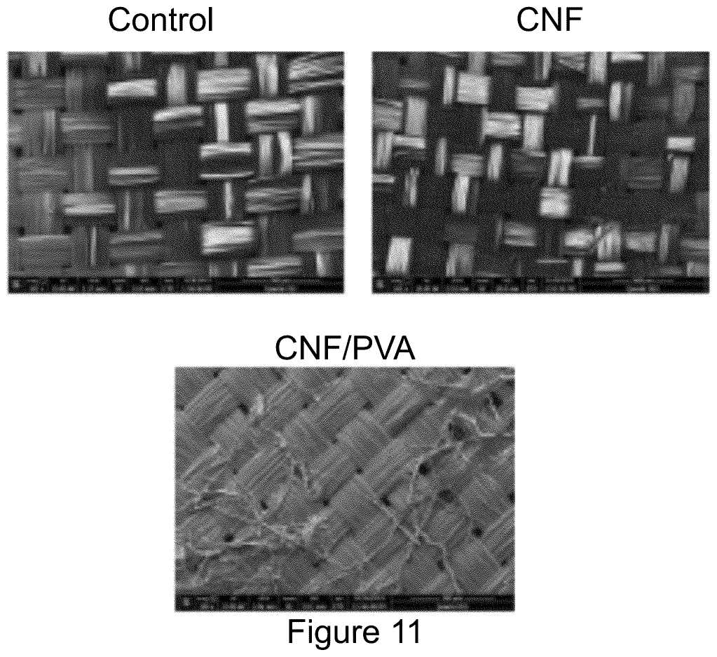

Photos of a control nylon fabric, nylon fabric sprayed with CNF, and nylon fabric sprayed with CNF/PVA were taken on an optical microscope and included 40× magnification, HDR 3-layered, and differential image contract (FIG. 11). Nylon fabric sprayed with CNF/PVA clearly depicted the presence of CNF in the porous areas of the fabric when compared to the control nylon fabric and nylon fabric sprayed with CNF. These results indicated that PVA acted as an adhering agent for CNF and there was a structural change on the surface of multiple textiles after the application of CNF/PVA.

### Example 3: Cellulose Nanofiber Water-Soluble Emulsion Composition—Chitosan (CS) Testing for Optimal Concentration

CS is the second most abundant polymer in nature behind cellulose, and is usually obtained from the deacetylation of chitin (Muley and Singhal, 2020, Zhang et al, 2021). This polysaccharide contains a variety of properties, which include good biocompatibility and degradability as well as advantageous antibacterial and film-forming properties (Sun et al, 2020).

CS modified into nanoparticles can enhance the charge interaction on the microorganism surface, which can create a desirable antibacterial effect (Pan et al, 2020). CS has been observed in previous publications in conjunction with CNF and PVA. These polymers were all hydrophilic, resulting in a simple combination to create the water-soluble emulsion. Specific to CS, soaking in acetic acid (pretreatment) followed by autoclaving can process long chain chitosan to short chain CS, which was a better option for combination with the CNF. This helped the CS to permeate into the pores of the CNF matrix to achieve a high loading content (Wei et al, 2021).

Additional studies tested three concentrations of CS with CNF/PVA to evaluate the most efficient concentration for antibacterial protection. Formulation I comprised 0.1% CS (0.101 g CS) in 100 mL of 1 wt % aqueous acetic acid solution (0.972 mL acetic acid, 100 mL H₂O). Formulation I was obtained by dissolving an initial measurement of 0.101 g of CS in 100 mL aqueous acetic acid solution (5% v/v) with continuous magnetic stirring (500 rpm) to obtain CS solution at room temperature (~25 °C). Then the CS was autoclaved at 115 °C for 30 minutes. Next, 20 mL of the CS solution was combined with the CNF and mixed until thoroughly homogenous.

Formulation II comprised 0.5% CS (0.504 g CS) in 100 mL of 1 wt % aqueous acetic acid solution (0.972 mL acetic acid, 100 mL H₂O). Formulation II was obtained by dissolving an initial measurement of 0.504 g of CS in 100 mL aqueous acetic acid solution (5% v/v) with continuous magnetic stirring (500 rpm) to obtain CS solution at room temperature (~25 °C). Then the CS was autoclaved at 115 °C for 30 minutes before it was combined with the CNF. Next, 12 mL of the CS solution was combined with the CNF and mixed until thoroughly homogenous.

Formulation III comprised 1% CS (1.013 g CS) in 100 mL of 1 wt % aqueous acetic acid solution (0.972 mL acetic acid, 100 mL H₂O). Formulation III was obtained by dissolving an initial measurement of 1.103 g of CS is dissolved in 100 mL aqueous acetic acid solution (5% v/v) with continuous magnetic stirring (500 rpm) to obtain CS solution at room temperature (~25 °C). Then the CS was autoclaved at 115 °C for 30 minutes before it was combined with the CNF. Next, 10 mL of the CS solution was combined with the CNF and mixed until thoroughly homogenous.

To generate a spray bottle with the most optimizable concentrations of CS, CNF, and PVA, ratios of (0.168%, 0.066%, 2.667%, or 0.840%, 0.066%, 2.667%, or 1.667%, 0.066%, 2.667%) were utilized in deionized water. The ratios pertained to pure concentrations of CS, CNF, and PVA. An initial measurement of (0.1 g, 0.5 g, or 1 g) of CS was dissolved in 100 mL aqueous acetic acid solution (1%, v/v) with continuous magnetic stirring (500 rpm) to obtain CS solution at room temperature (~25 °C). The CS was then autoclaved at 115 °C for 30 minutes.

Separately, the 3 wt % CNF were removed from the refrigerator at 1-4 °C and 1.35531 g was measured on the scale. CNF was added to the CS solution and homogenized at 13,000-17,000 rpm with a blender for 2 min and continued stirring with an IKA RW 20 stirrer (700 rpm) for 30 minutes.

Next, to prepare PVA, 5 g were measured on the scale and applied to a beaker with 50 mL of DI water. This created a weighted concentration of 9 wt % PVA, which was later applied to the water-soluble emulsion after heating. The solution was heated in a hot bath of 175 °C for 45-60 mins until the bubbles were completely diminished. 5 mL of the PVA solution was measured and distributed to the beaker containing CS and CNF. The CS, CNF, and PVA were placed into a 35 mL, 43 mL, or 45 mL beakers of DI water and spread evenly with an IKA RW 20 stirrer at a constant speed of 1000-2400 rpm. Once distributed into a 60-mL spray bottle, the solution was shaken well and ready to be sprayed. The test fabric samples were sprayed from one foot away with five layers and an appropriate minimum dry time of 1 hour before testing proceeded.

### Example 4: CNF Spray

Additional studies focused on further investigation of CS to determine its efficacy for the CNF spray.

As waste levels have soared dramatically in recent years, corrective measures have become essential to turn towards the usage of sustainable materials. Researchers are actively exploring alternative polymers that could supplant plastics and other fibrous substances, prioritizing those with analogous strength and chemical attributes. The Covid-19 pandemic underscored the importance of protection from bacterial aerosol transmission. Instead of relying solely on chemical antibacterial strategies for protection, researchers are steering towards sustainable avenues of defense.

Cellulose emerges as a pivotal option as a sustainable replacement to synthetic antibacterial agents, given its status as the most biodegradable, renewable, and recyclable biopolymer known. Build on the above studies that tested cellulose for its antibacterial properties following the biomimic concept, the herein described studies strengthen the antibacterial properties with an additional active ingredient.

The present studies focused on the investigation of supplementary active components, such as CS and silver nitrate, to bolster antibacterial resistance. Silver nitrate, already celebrated for its antibacterial prowess in applications like wound care and water purification, was chosen for its proven efficacy and to serve as a known control for the experiments. However, while silver nitrate is effective, its toxic potential when used in excess, which is why the use of natural antibacterial agents is necessary.

To evaluate the antibacterial properties of reagents, E. Coli was employed and its growth was monitored using the CGQ. E. Coli is a widely utilized bacterium in scientific research and has been largely cited in numerous peer-reviewed studies.

The CGQ, or Cellular Growth Quantifier, is an invaluable tool in bioprocess experimentation. It employs analytical technology for non-invasive, real-time biomass monitoring in shake flask cultures. The CGQ incorporates specialized sensors, primarily relying on backscatter light measurement, to incessantly monitor biomass within a culture vessel, eliminating the need for sample removal. This continuous data stream empowers researchers to observe microbial or cell culture growth phases closely, thereby gleaning insights into growth kinetics.

The herein described experiments used 250 mL shake flasks infused with varying reagents. Upon the addition of all ingredients, the flasks were housed in the CGQ for ~1 to 4 days. This window ensured sufficient time to accurately gauge the cell density corresponding to E. Coli growth within the flasks.

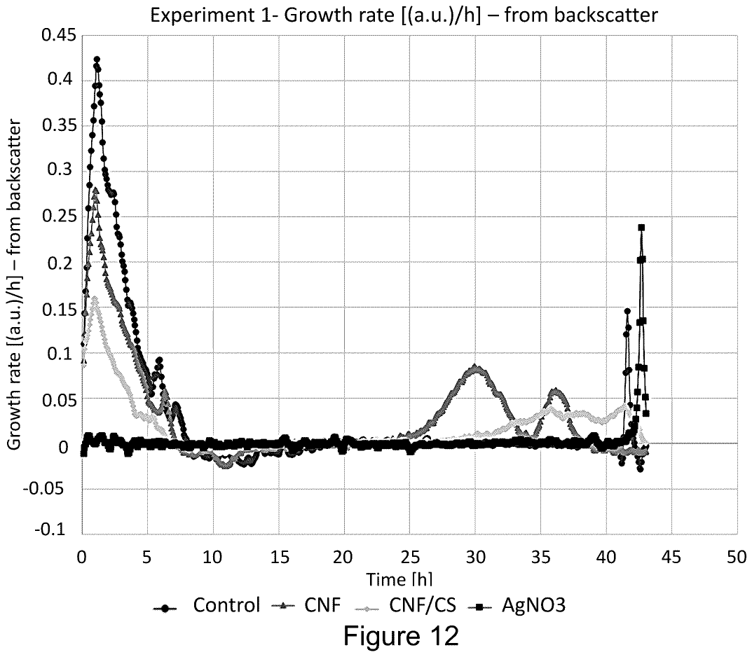

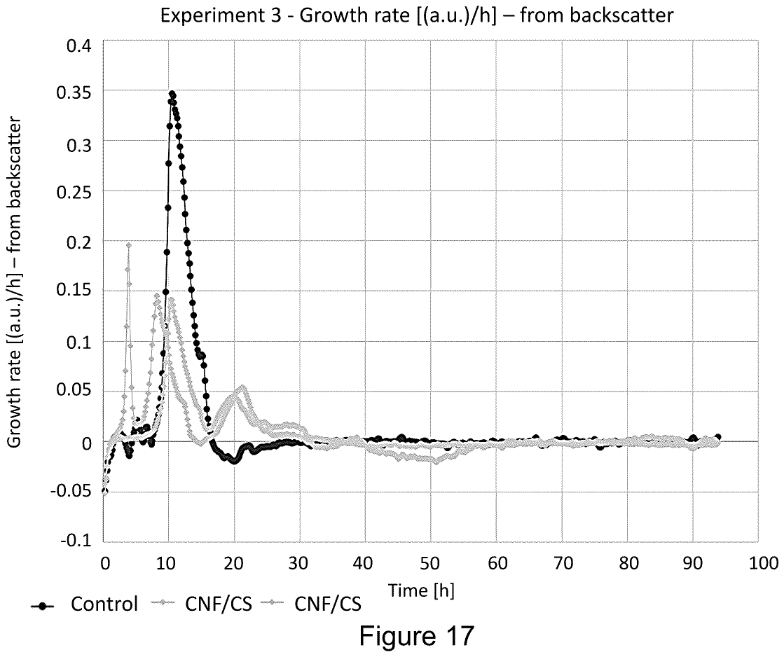

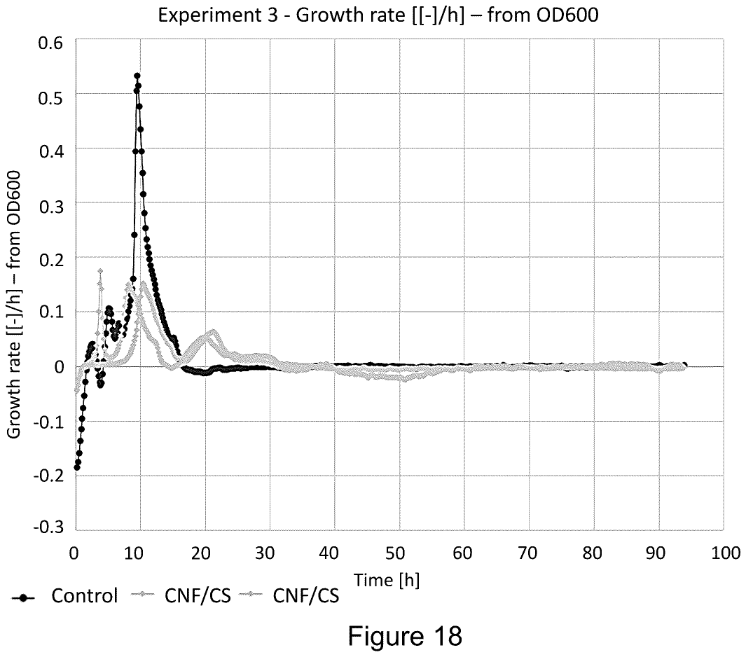

Representative growth rates of E. Coli were evaluated using both backscatter data and OD600 measurement methods (FIG. 12 through FIG. 15, FIG. 17, and FIG. 18). More specifically, for backscatter analysis, measurements via the CGQ indicated that backscattering assesses the light reflected back from the sample post-interaction with cells or particles. As the culture's cell density rises, the intensity of the scattered light, or backscatter signal, correspondingly amplifies. The y-axis labeled ‘Growth rate μ[1/h]’ outlines the pace of culture growth. In essence, the growth rate (commonly symbolized by ‘μ’) pinpoints the speed at which the cellular population augments.

For OD600 measurements, measurements were calibrated using the CGQ underscore that the acronym “OD600” defines Optical Density at the 600 nm wavelength. This metric is a standard method to ascertain cell density or concentration within a liquid culture. The technique hinges on the principle of light absorbance at the 600 nm wavelength as it traverses the culture. As cell density elevates, both scattering and absorption intensify, resulting in heightened OD600 values.

Both these techniques are geared towards shedding light on the growth dynamics of microbial or cell cultures, albeit through differing light interaction methods: scattering and absorbance.

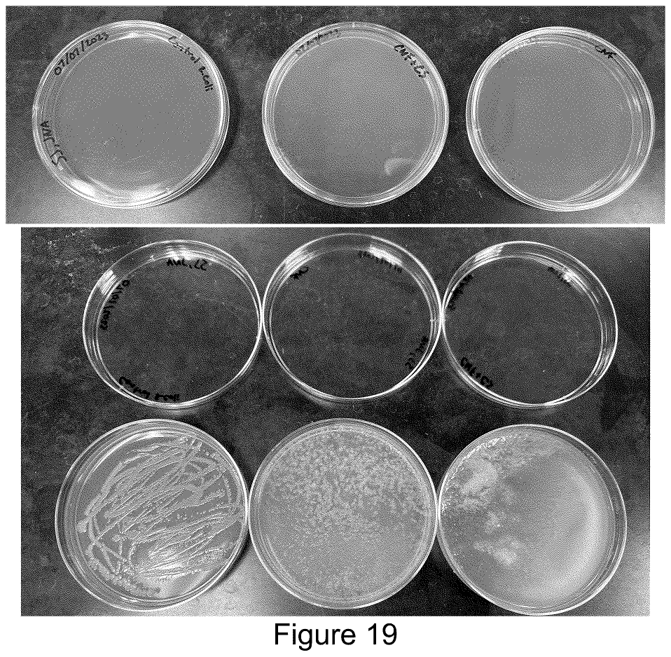

In addition to the CGQ method, agar plate tests were also conducted. Reagents were applied to the agar and incubated for a minimum of 72 hours (FIG. 19).

Experiment 1: Four 250 mL Flasks with Reagents Anticipated to Possess Antibacterial Properties (i.e., CNF, CS, and Silver Nitrate)

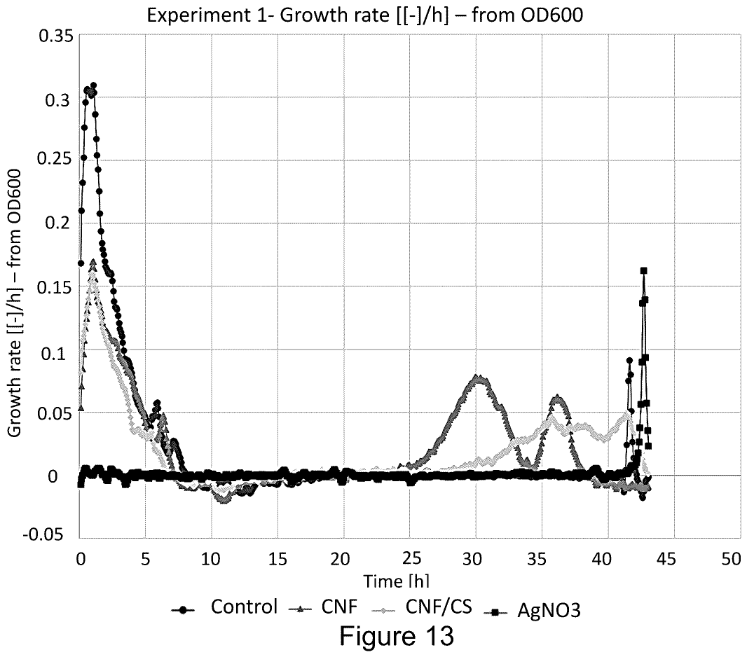

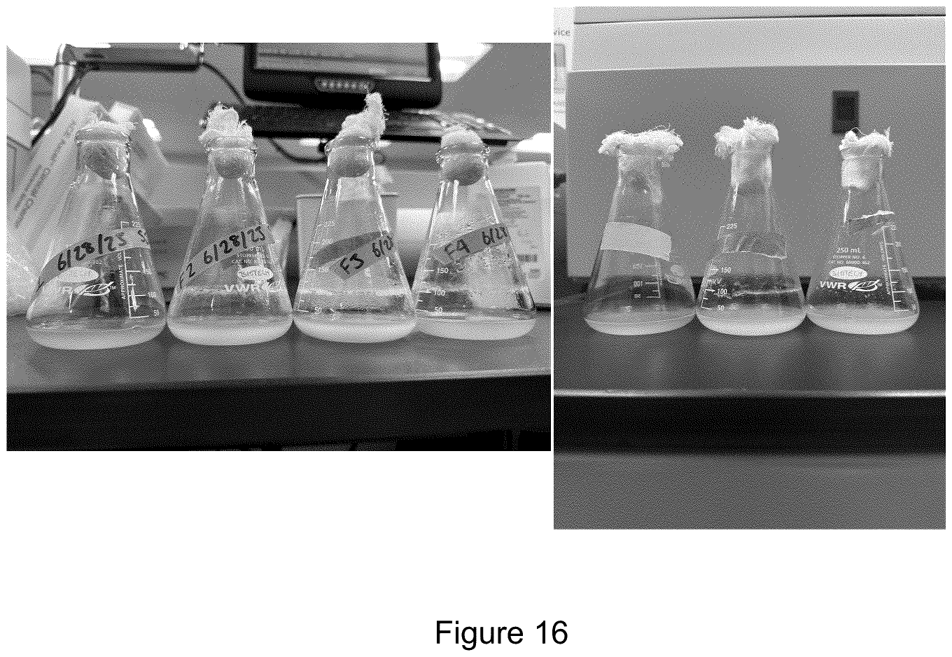

Experiment 1 evaluated four different solutions were prepared in 250 mL flasks and infused with ingredients anticipated to possess antibacterial properties. The CGQ was employed to conduct tests on these four 250 mL flasks (FIG. 16). Representative growth rates of E. Coli were evaluated using both backscatter data and OD600 measurement methods (FIG. 12 and FIG. 13).

More specifically, FIG. 12 depicts a representative the rate at which the E. coli culture is growing with the x-axis of time and a y-axis of Growth rate μ[1/h]. A heightened point was indicative of a more populated cell culture. The Control flask, devoid of additives, contained a maximum peak of coordinates (1.04, 0.42). Additionally, the flask with only CNF contained a maximum peak of coordinates at (0.91, 0.28). Intriguingly, merging CNF with CS curtailed the growth rate, climaxing at (0.78, 0.16). This indicated a combinatory inhibitory action of CNF and CS, indicating an enhanced antibacterial mechanism with the CS addition. In contrast, the silver nitrate results, with the lowest maximum value of (1.42, 0.011), demonstrated barely any growth of the E. Coli. The variable growth rates accentuated the distinct antibacterial efficacy of the additives.

FIG. 13 depicts a graphical representation plotting time on the x-axis and the growth rate μ[1/h], derived from OD600 measurements, on the y-axis, illustrating the progression of cell culture expansion. A pronounced peak indicated denser cell concentrations with light absorbance at the 600 nm wavelength. The control flask, containing no additives, peaked at (0.04, 0.31). The flask infused solely with CNF achieved a maximum at (0.91, 0.17). The combination of CNF with CS exhibited a similar peak to the CNF-alone flask, reaching a high of (0.91, 0.16). This subtle difference indicated an increased antibacterial property between CNF and CS concerning bacterial growth inhibition. The silver nitrate experimental flask showcased the least cell growth, peaking at a notably low (1.42, 0.01). These varied peaks emphasized the distinct influence of each additive on E. Coli growth, highlighting the potential and effectiveness of the chitosan addition these agents in antibacterial applications.

Experiment 2: Three 250 mL Flasks with Reagents Anticipated to Possess Antibacterial Properties (i.e., CNF)

In the following experimental procedures, to achieve more clear results, E. Coli were grown directly from the flask itself without an initial starter culture to identify if CNF and/or CS affected the E. Coli growth from the first states of cellular division.

Three flasks for each procedure were used to have one control and two experimental flasks, and one colony of E. Coli (FIG. 16). The two experimental flasks contained the same exact composition with identical amounts of CNF for a more direct comparison.

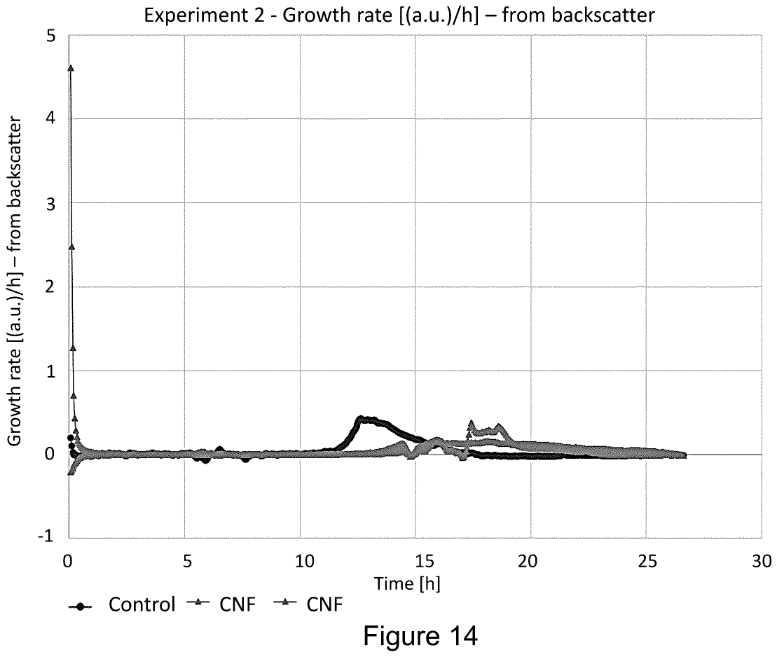

FIG. 14 portrays the progression of time on the x-axis compared with the growth rate μ[1/h] on the y-axis. These measurements offered insights into the rapidity of culture growth: higher points on the y-axis denote increased growth rates, indicating less effective antibacterial activity, while lower points implied inhibited bacterial growth due to the presence of antibacterial agents. The control flask, without any additives, exhibited a growth rate peaking at (12.54, 0.54). This served as the baseline. Both experimental flasks infused with CNF contained reduced growth rates, peaking at (17.35,0.48) and (17.99, 0.30), respectively.

This decline in growth rates in the presence of CNF indicated CNF's efficacy in suppressing E. Coli proliferation. The discrepancy in growth rates between the control and the CNF-laden flasks underscored the potential antibacterial properties of CNF. Given the reduced rates in the experimental flasks, it can be inferred that CNF, began influencing the very initial stages of E. Coli cellular division, reiterating the statemen of CNF as a prospective antibacterial agent.

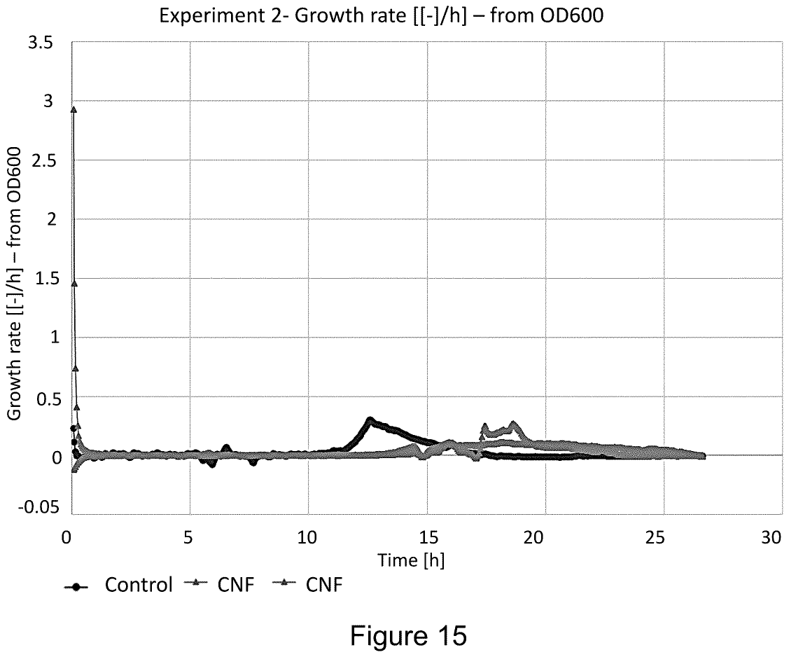

FIG. 15 depicts a graphical representation of the cell growth where the x-axis plots the duration of culture growth, while the y-axis denotes the growth rate μ[1/h], derived from the OD600 measurements. OD600, or Optical Density at 600 nm, quantifies the cell density in a liquid culture by gauging light absorbance at the 600 nm wavelength. Essentially, a spike in OD600 values implies heightened cell density, attributable to increased light scattering and absorption by proliferating cells.

The control flask, devoid of additives, showcased a growth rate that peaked at (12.52,0.33). This provided a point of reference for the inherent growth potential of the E. Coli under normal conditions. Contrarily, the experimental flasks infused with CNF recorded suppressed growth rates, reaching peaks at (18.68,0.29) and (18.04, 0.12), respectively.

Notably, the considerable dip in growth rates articulated the inhibitory action of CNF on E. Coli proliferation. The CNF's evident impact, marked by reduced growth rates even from the outset of cellular division, reinforced its standing as a promising antibacterial agent.

Experiment 3: Three 250 mL Flasks with Reagents Anticipated to Possess Antibacterial Properties (i.e., CNF and CS)

Additional procedures consisted of three flasks: one control and two experimental. The two experimental flasks contained identical amounts of CNF and CS, and one colony of E. Coli.

As shown in FIG. 17, the E. coli growth rate data was plotted with time on the x-axis and the growth rate μ[1/h] on the y-axis, derived from backscattering measurements. The technique of backscattering quantifies the light redirected from the sample post its interaction with cells or particulates. Within the realm of cell cultures, an increased cell density was indicative of enhanced light scattering, leading to a heightened backscatter signal.

The control flask, free from any additives, displayed a growth rate peaking at coordinates (1.02, 0.35). This served as a baseline for E. Coli growth without the influence of external agents. In contrast, the experimental flasks infused with the combination of CNF and CS, presented subdued growth rates. Their peaks were located at (10.21,0.14) and (3.63, 0.19), respectively. These observations highlighted the inhibitory nature of the CNF and CS blend on E. Coli growth. Both experimental flasks demonstrated a large decrease in growth rate, further indicating that a synergy between CNF and CS intensified their antibacterial efficacy.

In the context of both graphs from Experiment 2 and Experiment 3 (FIG. 14 and FIG. 17), the purpose was to measure the impact of CNF in isolation and later in combination with CS on E. Coli growth through the CGQ. Both experiments consistently plotted time against the growth rate μ[1/h]. Upon further examination of the results, E. Coli's growth decreased in the presence of CNF, evident in Experiment 2 (FIG. 14). Here, the growth rate's maxima decreased from the control's 0.54 measured value to 0.48 and then to a lower 0.30 with CNF only.

In contrast, Experiment 3's introduction of CS alongside CNF amplified this inhibitory action (FIG. 17). The recorded peak growth rates dropped further to 0.14 and 0.19, from a control value of 0.35. This progression underscored a two-tiered inhibitory effect: initially by CNF, then further emphasized by the joint combination of CNF and CS, strengthening their potential as significant assets in curbing bacterial growth.

Furthermore, as shown in FIG. 18, three flasks were prepared to evaluate the effects of CNF and CS on E. Coli growth, using the OD600 method to gauge cell density in liquid culture. The control flask, without any additives, peaked at a growth rate of (9.21,0.53). This provided a baseline indicating the natural growth rate of E. Coli in the absence of external agents. Contrastingly, the experimental flasks infused with a blend of CNF and CS painted a different picture. One of the experimental flasks exhibited a growth rate peak at (3.65,0.17) and, similarly, the other experimental flask showed a peak at (10.27,0.15). These subdued growth rates strongly highlighted the inhibitory effects of CS when combined with CNF. The observed variations in growth rates reiterated the potent antibacterial properties of CS and its potential combinatory effects with CNF.

When these findings from both Experiment 2 and Experiment 3 were combined, a clear synergistic effect was present (FIG. 15 and FIG. 18). In Experiment 2, E. Coli's growth, in the absence of any additives, had a peak growth rate of 0.33 (FIG. 15). However, with the introduction of CNF, the growth rates dipped to 0.29 and as low as 0.12. Transitioning to Experiment 3, where both CNF and CS were applied, E. Coli growth in the control flask showcased a higher peak growth rate of 0.53 (FIG. 18). However, in the presence of the combined CNF and CS, the growth rates decreased even further, recording values of 0.17 and 0.15. These results clearly indicated that while CNF on its own moderated E. Coli growth, the incorporation of CS amplified this inhibitory effect. These findings strongly indicated the CS's antibacterial properties, especially when combined with CNF, positioning them as important reagents in antibacterial research.

Experiment 4: E. Coli Growth on Agar Plates with Deposited CNF and CS

Additional studies aimed to examine the influence of CNF and CS on the growth of E. Coli on agar plates. Distinct variations in bacterial proliferation patterns were observed across the different test conditions (FIG. 19). The control plate, devoid of any additives, showcased vivid E. Coli colony formation, as depicted by clear, well-defined colonies and the characteristic streaking patterns resulting from the plate streaking method (FIG. 19, left column). This followed the natural E. Coli growth behavior under unaltered conditions.

Conversely, the plate supplemented solely with CNF exhibited a significant alteration in E. Coli growth patterns (FIG. 19, middle column). Rather than forming distinct colonies reminiscent of the control, bacterial presence was diffusely scattered across the plate, lacking the structure and consistency associated with regular colony formation. Furthermore, the possible residues of CNF might have been left on the plate, indicating potential interactions between the bacteria and CNF or incomplete dissolution of the CNF.

Most importantly, the plate infused with both CNF and CS demonstrated the most dramatic shift from the control (FIG. 19, right column). The patterns observed were erratic and uneven, indicating a profound influence of the combined CNF and CS additives. Within these atypical growth patterns, E. Coli was spotted mainly on the top left of the plate. Additionally, this plate was notably moister than the other agar plates, with residual liquid on the bottom right. This excess moisture could be from the doubled volume of the reagents (5 mL in total) compared to the other plates which received 2.5 mL or less, potentially impacting the bacterial growth and interaction with the additives.

In total, these results underscored the significant impact of CNF, especially when coupled with CS, on impeding regular E. Coli growth. The observed patterns, particularly middle and right plates, depicted these compounds as influential agents in microbial control (FIG. 19). Additional investigations evaluate the exact mechanisms by which CNF and CS affect bacterial growth and optimize their concentrations for desired effects.

The materials and methods employed in the present experimental example are now described.

### Materials

Reagents used included: CNF Slurry 3.0 wt %aqueous gel, fiber width of 50 nm, lengths of up to several hundred microns, Silver Nitrate: AgNO₃, ≥99.0% titration, CS with mean molecular weight of 100,000 Da, viscosity of 20-300 cP, and degree of deacetylation of 80-90%, E. Coli, HB101 strain cultured in lysogeny broth (LB broth), LB broth, and HB 101 plate with colonies present.

Lab tools used included: inoculating loop, fume hood, pop cap tubes, pop cap small containers, weighted scale, and beakers.

### Methods

Preparation of E. Coli cultures used in Experiment 1: E. Coli was prepared 16 hours before use to achieve an adequate culture. A plate of B101 with at least 4 colonies was obtained before experimentation proceeded. 5 mL of broth was distributed each into 4 pop cap miniature tubes. Using an aseptic technique, a colony on the plate of HB 101 colonies was scooped with a sterile inoculating loop and put in the 5 mL tube. The inoculating loop was twisted around 5 times to dispense the cells in the tube. This step was performed separately for each of the 4 pop cap tubes. Subsequently transfer the 4 pop cap tubes into the shaking incubator and leave the tubes overnight to grow at 250 rpm.

Preparation of E. Coli cultures used in Experiment 2 and Experiment 3: LB broth was removed from the fridge allowed to warm up to room temperature. Plate of HB 101 with at least 3 colonies for experimentation was removed from the fridge. The flasks were autoclaved before beginning the procedure. During the procedure, 28 mL of broth was initially distributed into each flask. Three separate inoculating loops were obtained. A colony on the plate of HB 101 colonies was scooped with a sterile inoculating loop using an aseptic technique scoop and placed in each flask. The inoculating loop was twisted around 5 times to dispense the cells in the tube. This step was performed separately for each of the 3 flasks. The flasks were then covered with the cotton and cheese cloth before distributing other materials.

Preparation of CNF: 3 wt % CNF was stored at 1-4 °C. 1.35531 g of 3 wt % CNF was measured in room temperature under ambient air and left at room temperature until distributed into the designated beaker.

Preparation of Silver Nitrate: 0.00413876 g of silver nitrate was measured and kept in a pop cap small container until distributed into the final solution.

Preparation of CS: 280 mg of CS was measured and kept in a pop cap small container until distributed into the final solution.

Preparation of the Individual 250 mL Flasks for Experiment 1: A cotton ball and cheese cloth were wrapped and put into the opening of the flask. The opening was subsequently wrapped with foil and an autoclave indicator tape was placed on the foil. The 250 mL flasks were autoclaved in the wrapped cycle for around 45 minutes. The foil wrapping was subsequently removed.

Flask 1 for Experiment 1: 23 mL of the LB broth was dispensed into the 250 mL beaker, followed by 5 mL of the grown E. Coli culture. The mixture was swirled to generate a homogenous mixture.

Flask 1 for Experiment 2 and Experiment 3: 28 mL of the LB broth was dispensed into the 250 mL beaker, followed by the E. Coli culture dispensed with an inoculation loop. The mixture was swirled to generate a homogenous mixture.

Flask 2 for Experiment 1: 23 mL of the LB broth was dispensed into the 250 mL beaker, followed by 5 mL of the grown E. Coli culture. 1.355 g of CNF was subsequently added to the beaker. The mixture was swirled to generate a homogenous mixture.

Flask 2 for Experiment 2: 28 mL of the LB broth was dispensed into the 250 mL beaker, followed by the E. Coli culture dispensed with an inoculation loop. 1.355 g of CNF was subsequently added to the beaker. The mixture was swirled to generate a homogenous mixture.

Flask 2 for Experiment 3: 28 mL of the LB broth was dispensed into the 250 mL beaker, followed by the E. Coli culture dispensed with an inoculation loop. 1.355 g of CNF and then 280 mg CS were subsequently added to the beaker. The mixture was swirled to generate a homogenous mixture.

Flask 3 for Experiment 1: 23 mL of the LB broth was dispensed into the 250 mL beaker, followed by 5 mL of the grown E. Coli culture. 1.355 g of CNF and then 280 mg of CS were subsequently added to the beaker. The mixture was swirled to generate a 1 wt % homogenous mixture.

Flask 3 for Experiment 2: 28 mL of the LB broth was dispensed into the 250 mL beaker, followed by the E. Coli culture dispensed with an inoculation loop. 1.355 g of CNF was subsequently added to the beaker. The mixture was swirled to generate a homogenous mixture.

Flask 3 for Experiment 3: 28 mL of the LB broth was dispensed into the 250 mL beaker, followed by the E. Coli culture dispensed with an inoculation loop. 1.355 g of CNF and then 280 mg CS were subsequently added to the beaker. The mixture was swirled to generate a homogenous mixture.

Flask 4 for Experiment 1: 23 mL of the LB broth was dispensed into the 250 mL beaker, followed by 5 mL of the grown E. Coli culture. 1.355 g of CNF and then 5.14 mg AgNO₃ were subsequently added to the beaker. The mixture was swirled to generate a homogenous mixture.

Experiment 1, Implementation of the 250 mL Beakers into the CGQ was achieved by utilizing the CGQuant application and inputting the following information: in total, each flask can hold up to 250 mL, the filled volume is 29 mL for each flask, and the shaking incubator moves at 150 rpm. The same procedure was followed for all flasks.

Experiment 2 and Experiment 3, Implementation of the 250 mL Beakers into the CGQ was achieved by utilizing the CGQuant application and inputting the following information: in total, each flask can hold up to 250 mL, the filled volume is 29 mL for each flask, except control, which is 28 mL, the shaking incubator moves at 150 rpm, and temperature between 30 to 40 °C. The same procedure was followed for all flasks.

Experiment 4: Plate 1 (control) contained a smeared E. Coli colony, plate 2 (experimental CNF) contained a smeared E. Coli colony and 0.5 g of CNF in 2.5 mL of water (0.5 wt %), and plate 3 (experimental CNF+CS) contained smeared E. Coli colony, 0.5 g of CNF in 2.5 mL of water (0.5 wt %), and 0.5 g of CS in 2.5 mL of water (16.67 wt %).

Agar Plates Procedure: Initially, each agar plate was smeared with one E. Coli bacterial colony. After that, a 5 mL stereological pipette was used to add 2.5 mL of CNF to plate 2, covering the whole plate. A 5 mL serological pipette was then used to add 2.5 mL of CNF and 2.5 mL of CS to cover the whole plate 3. Once deposited, the agar plates were covered with lids and incubated at 37 °C for approximately 72 hours.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition for increasing filtration efficiency,
wherein the composition comprises cellulose nanofiber (CNF) or a derivative thereof, an adhering agent, and a carrier.

2. The composition of claim 1, wherein the composition comprises between about 0.01 wt % to about 20 wt % of CNF, between about 0.01 wt % to about 20 wt % of the adhering agent, or a combination thereof.

3.-5. (canceled)

6. The composition of claim 1, wherein
a)the adhering agent comprises at least one polymer;
b) the adhering agent specifically binds CNF to a surface of a material of interest; or
c) a combination thereof.

7.-8. (canceled)

9. The composition of claim 6, wherein the polymer is selected from the group consisting of a polyhydroxy polymer, water-soluble polymer, biodegradable polymer, biocompatible polymer, polyvinyl alcohol (PVA), polyethylene glycol (PEG), 1,2-distearoyl-sn-glycero-3-phosphoethanolamine-polyethylene glycol (DSPE-PEG) 1,2-distearoyl-sn-glycero-3-phosphoethanolamine-N-[amino (polyethylene glycol)-20001 (DSPE-PEG (2000)-amine), and any combination thereof.

10-14. (canceled)

15. The composition of claim 1, wherein the carrier comprises an aqueous solution.

16.-17. (canceled)

18. The composition of claim 1, wherein the composition is a water-soluble composition, biodegradable composition, biocompatible composition, antimicrobial composition, antibacterial composition, antiviral composition, antifungal composition, or any combination thereof.

19. The composition of claim 1, wherein
a) the composition increases barrier properties against particles,
b) the composition breaks a membrane of at least one cell of interest: or
c) a combination thereof.

20. The composition of claim 10, wherein
a)the particles have an average hydrodynamic diameter of about 0.1 nm to about 100,000 nm;
b) the particles are aerosol particles; or
c) a combination thereof.

21.-25. (canceled)

26. The composition of claim 1, wherein the composition further comprises at least one therapeutic agent.

27. The composition of claim 26, wherein
a) the composition comprises between about 0.01 wt % to about 20 wt % of the at least one therapeutic agent;
b) the at least one therapeutic agent is selected from the group consisting of antimicrobial agent, antibacterial agent, antiviral agent, antifungal agent, chitosan (CS) or a derivative thereof, sodium alginate (SA) or a derivative thereof and any combination thereof; or
c) a combination thereof.

28.-32. (canceled)

33. A method of increasing barrier properties of a material of interest against at least one particle,
wherein the method comprises applying at least one layer of the composition of claim 1 to the surface of the material of interest.

34. The method of claim 33, wherein the composition
a) decreases a contact angle between the at least one particle and the material of interest;
b) decreases a size of at least one pore of the material of interest; or
c) a combination thereof.

35. The method of claim 33, wherein
a) the particles have an average hydrodynamic diameter of about 0.1 nm to about 100,000 nm;
bi the particles are aerosol particles; or
c) a combination thereof.

36.-37. (canceled)

38. The method of claim 33, wherein the material of interest is selected from the group consisting of a textile material, paper, glass, metal, wood, plastic, and any combination thereof.

39. (canceled)

40. A method of applying at least one composition of claim 1 to a material of interest,
wherein the method comprises applying at least one layer of the composition to the surface of the material of interest.

41. The method of claim 40, wherein the material of interest is selected from the group consisting of a textile material, paper, glass, metal, wood, plastic, and any combination thereof.

42. (canceled)

43. The method of claim 40, wherein the at least one layer of the composition is applied to the surface of the material of interest by spraying.

44. (canceled)

45. A device comprising a material of interest and the composition of claim 1, wherein the device comprises at least one layer of the composition coated on the surface of the material of interest.

46. The device of claim 45, wherein the material of interest is selected from the group consisting of a textile material, paper, glass, metal, wood, plastic, and any combination thereof.

47. (canceled)

48. The device of claim 45, wherein
a) the device increases barrier properties against particles:
b) the device is a mask, clothing, fabric, textile furniture carpet curtain upholstery, filter, or any combination thereof; or
c) any combination thereof.

49. (canceled)
